SEQUENCE LISTING (18 sequences)

SEQ ID NO: 1 - protein, 1360 aa, synthetic construct
  REGION 1..1360: note = consensus sequence
  VARIANT 6..10: note = Xaa can be any naturally occurring amino acid
  VARIANT 17..22: note = Xaa can be any naturally occurring amino acid
  VARIANT 30..34: note = Xaa can be any naturally occurring amino acid
  VARIANT 48..52: note = Xaa can be any naturally occurring amino acid
  VARIANT 54..59: note = Xaa can be any naturally occurring amino acid
  VARIANT 81..84: note = Xaa can be any naturally occurring amino acid
  VARIANT 89..95: note = Xaa can be any naturally occurring amino acid
  VARIANT 100..102: note = Xaa can be any naturally occurring amino acid
  VARIANT 105..106: note = Xaa can be any naturally occurring amino acid
  VARIANT 114..116: note = Xaa can be any naturally occurring amino acid
  VARIANT 120..121: note = Xaa can be any naturally occurring amino acid
  VARIANT 142..143: note = Xaa can be any naturally occurring amino acid
  VARIANT 145..146: note = Xaa can be any naturally occurring amino acid
  VARIANT 183..184: note = Xaa can be any naturally occurring amino acid
  VARIANT 186..187: note = Xaa can be any naturally occurring amino acid
  VARIANT 233..234: note = Xaa can be any naturally occurring amino acid
  VARIANT 265..267: note = Xaa can be any naturally occurring amino acid
  VARIANT 269..270: note = Xaa can be any naturally occurring amino acid
  VARIANT 273..274: note = Xaa can be any naturally occurring amino acid
  VARIANT 305..307: note = Xaa can be any naturally occurring amino acid
  VARIANT 313..314: note = Xaa can be any naturally occurring amino acid
  VARIANT 321..325: note = Xaa can be any naturally occurring amino acid
  VARIANT 328..329: note = Xaa can be any naturally occurring amino acid
  VARIANT 333..336: note = Xaa can be any naturally occurring amino acid
  VARIANT 410..411: note = Xaa can be any naturally occurring amino acid
  VARIANT 446..449: note = Xaa can be any naturally occurring amino acid
  VARIANT 479..481: note = Xaa can be any naturally occurring amino acid
  VARIANT 565..566: note = Xaa can be any naturally occurring amino acid
  VARIANT 572..573: note = Xaa can be any naturally occurring amino acid
  VARIANT 671..673: note = Xaa can be any naturally occurring amino acid
  VARIANT 681..683: note = Xaa can be any naturally occurring amino acid
  VARIANT 751..752: note = Xaa can be any naturally occurring amino acid
  VARIANT 754..756: note = Xaa can be any naturally occurring amino acid
  VARIANT 798..800: note = Xaa can be any naturally occurring amino acid
  VARIANT 807..813: note = Xaa can be any naturally occurring amino acid
  VARIANT 816..817: note = Xaa can be any naturally occurring amino acid
  VARIANT 819..821: note = Xaa can be any naturally occurring amino acid
  VARIANT 823..827: note = Xaa can be any naturally occurring amino acid
  VARIANT 841..842: note = Xaa can be any naturally occurring amino acid
  VARIANT 856..859: note = Xaa can be any naturally occurring amino acid
  VARIANT 924..927: note = Xaa can be any naturally occurring amino acid
  VARIANT 929..930: note = Xaa can be any naturally occurring amino acid
  VARIANT 932..933: note = Xaa can be any naturally occurring amino acid
  VARIANT 937..938: note = Xaa can be any naturally occurring amino acid
  VARIANT 963..965: note = Xaa can be any naturally occurring amino acid
  VARIANT 981..982: note = Xaa can be any naturally occurring amino acid
  VARIANT 995..997: note = Xaa can be any naturally occurring amino acid
  VARIANT 1020..1021: note = Xaa can be any naturally occurring amino acid
  VARIANT 1041..1042: note = Xaa can be any naturally occurring amino acid
  VARIANT 1105..1106: note = Xaa can be any naturally occurring amino acid
  VARIANT 1213..1214: note = Xaa can be any naturally occurring amino acid
  VARIANT 1231..1235: note = Xaa can be any naturally occurring amino acid
  VARIANT 1240..1246: note = Xaa can be any naturally occurring amino acid
  VARIANT 1255..1256: note = Xaa can be any naturally occurring amino acid
  VARIANT 1260..1263: note = Xaa can be any naturally occurring amino acid
  VARIANT 1265..1266: note = Xaa can be any naturally occurring amino acid
  VARIANT 1268..1270: note = Xaa can be any naturally occurring amino acid
  VARIANT 1272..1273: note = Xaa can be any naturally occurring amino acid
  VARIANT 1277..1278: note = Xaa can be any naturally occurring amino acid
  VARIANT 1280..1281: note = Xaa can be any naturally occurring amino acid
  VARIANT 1283..1284: note = Xaa can be any naturally occurring amino acid
  VARIANT 1298..1299: note = Xaa can be any naturally occurring amino acid
  VARIANT 1320..1321: note = Xaa can be any naturally occurring amino acid
  VARIANT 1325..1328: note = Xaa can be any naturally occurring amino acid
  VARIANT 1345..1348: note = Xaa can be any naturally occurring amino acid
  VARIANT 1350..1351: note = Xaa can be any naturally occurring amino acid
  SITE 12: note = Xaa can be any naturally occurring amino acid
  SITE 14: note = Xaa can be any naturally occurring amino acid
  SITE 26: note = Xaa can be any naturally occurring amino acid
  SITE 28: note = Xaa can be any naturally occurring amino acid
  SITE 46: note = Xaa can be any naturally occurring amino acid
  SITE 79: note = Xaa can be any naturally occurring amino acid
  SITE 86: note = Xaa can be any naturally occurring amino acid
  SITE 111: note = Xaa can be any naturally occurring amino acid
  SITE 124: note = Xaa can be any naturally occurring amino acid
  SITE 127: note = Xaa can be any naturally occurring amino acid
  SITE 134: note = Xaa can be any naturally occurring amino acid
  SITE 136: note = Xaa can be any naturally occurring amino acid
  SITE 140: note = Xaa can be any naturally occurring amino acid
  SITE 157: note = Xaa can be any naturally occurring amino acid
  SITE 206: note = Xaa can be any naturally occurring amino acid
  SITE 208: note = Xaa can be any naturally occurring amino acid
  SITE 210: note = Xaa can be any naturally occurring amino acid
  SITE 213: note = Xaa can be any naturally occurring amino acid
  SITE 245: note = Xaa can be any naturally occurring amino acid
  SITE 299: note = Xaa can be any naturally occurring amino acid
  SITE 316: note = Xaa can be any naturally occurring amino acid
  SITE 361: note = Xaa can be any naturally occurring amino acid
  SITE 373: note = Xaa can be any naturally occurring amino acid
  SITE 420: note = Xaa can be any naturally occurring amino acid
  SITE 426: note = Xaa can be any naturally occurring amino acid
  SITE 428: note = Xaa can be any naturally occurring amino acid
  SITE 466: note = Xaa can be any naturally occurring amino acid
  SITE 486: note = Xaa can be any naturally occurring amino acid
  SITE 492: note = Xaa can be any naturally occurring amino acid
  SITE 497: note = Xaa can be any naturally occurring amino acid
  SITE 570: note = Xaa can be any naturally occurring amino acid
  SITE 581: note = Xaa can be any naturally occurring amino acid
  SITE 586: note = Xaa can be any naturally occurring amino acid
  SITE 598: note = Xaa can be any naturally occurring amino acid
  SITE 605: note = Xaa can be any naturally occurring amino acid
  SITE 616: note = Xaa can be any naturally occurring amino acid
  SITE 618: note = Xaa can be any naturally occurring amino acid
  SITE 626: note = Xaa can be any naturally occurring amino acid
  SITE 665: note = Xaa can be any naturally occurring amino acid
  SITE 667: note = Xaa can be any naturally occurring amino acid
  SITE 700: note = Xaa can be any naturally occurring amino acid
  SITE 705: note = Xaa can be any naturally occurring amino acid
  SITE 709: note = Xaa can be any naturally occurring amino acid
  SITE 715: note = leucine (Leu) or methionine (Met)
  SITE 721: note = Xaa can be any naturally occurring amino acid
  SITE 737: note = Xaa can be any naturally occurring amino acid
  SITE 741: note = Xaa can be any naturally occurring amino acid
  SITE 744: note = Xaa can be any naturally occurring amino acid
  SITE 760: note = Xaa can be any naturally occurring amino acid
  SITE 763: note = Xaa can be any naturally occurring amino acid
  SITE 796: note = Xaa can be any naturally occurring amino acid
  SITE 802: note = Xaa can be any naturally occurring amino acid
  SITE 804: note = Xaa can be any naturally occurring amino acid
  SITE 831: note = Xaa can be any naturally occurring amino acid
  SITE 861: note = Xaa can be any naturally occurring amino acid
  SITE 863: note = Xaa can be any naturally occurring amino acid
  SITE 886: note = Xaa can be any naturally occurring amino acid
  SITE 895: note = Xaa can be any naturally occurring amino acid
  SITE 903: note = Xaa can be any naturally occurring amino acid
  SITE 920: note = Xaa can be any naturally occurring amino acid
  SITE 950: note = Xaa can be any naturally occurring amino acid
  SITE 957: note = Xaa can be any naturally occurring amino acid
  SITE 959: note = Xaa can be any naturally occurring amino acid
  SITE 972: note = Xaa can be any naturally occurring amino acid
  SITE 975: note = Xaa can be any naturally occurring amino acid
  SITE 978: note = Xaa can be any naturally occurring amino acid
  SITE 1025: note = Xaa can be any naturally occurring amino acid
  SITE 1076: note = Xaa can be any naturally occurring amino acid
  SITE 1080: note = Xaa can be any naturally occurring amino acid
  SITE 1095: note = Xaa can be any naturally occurring amino acid
  SITE 1112: note = Xaa can be any naturally occurring amino acid
  SITE 1115: note = Xaa can be any naturally occurring amino acid
  SITE 1140: note = Xaa can be any naturally occurring amino acid
  SITE 1144: note = Xaa can be any naturally occurring amino acid
  SITE 1165: note = Xaa can be any naturally occurring amino acid
  SITE 1181: note = Xaa can be any naturally occurring amino acid
  SITE 1185: note = Xaa can be any naturally occurring amino acid
  SITE 1194: note = Xaa can be any naturally occurring amino acid
  SITE 1197: note = Xaa can be any naturally occurring amino acid
  SITE 1258: note = Xaa can be any naturally occurring amino acid
  SITE 1275: note = Xaa can be any naturally occurring amino acid
  SITE 1295: note = Xaa can be any naturally occurring amino acid
  SITE 1315: note = Xaa can be any naturally occurring amino acid
  SITE 1323: note = Xaa can be any naturally occurring amino acid
  SITE 1330: note = Xaa can be any naturally occurring amino acid
  SITE 1336: note = Xaa can be any naturally occurring amino acid
  SITE 1339: note = Xaa can be any naturally occurring amino acid
  SITE 1341: note = Xaa can be any naturally occurring amino acid
  SITE 1343: note = Xaa can be any naturally occurring amino acid
  SITE 1359: note = Xaa can be any naturally occurring amino acid
MKEKRXXXXXPXVXPKXXXXXXWDDXDXPXXXXXIKRQSIDHGVGXEXXXXXEXXXXXXFRKYNSQGFKAKDTDLMGTXLXXXXEXELXXXXXXXADQQXXXSAXXERKKXPTXXXLDPXXEPXIFXILDTDYXIXPAQXVXXGXXDTEDGKPVIRXFGKKENGEFKIEYDRTFEPYFYALLXXGXXKDDLSAIEEVKKITAERHXRXGXETXVRVVDAEEVVKKFEPLGLEXXRPIEVWKLYFXHPQDVPAIRDKIREHPAVVXXXGXXDIXXYEYDIPFAKRYLIDKGLIPPMSWVXMEGDKXXXGRLLNXXSXCQLEXXXXXSDXXVHPXXXXRLEELPDYIPPLKVLAFDIETLYHXELGSFPEPGKGXRPIIAITHYDSIDDRFYVFDMISSAYADEEEARPFLXXVITWKNIDXLPYEIXDXVDVVSFDTEREMLKRFLXXXXEKDPDVIIGYNGDNFDXPYLKKRAEKLKVXXXGIPLXLGRLSXKRSSXDGSVFEPKIQRMYGDRFAVEICLGYKGKGRIHFDLYPVIRRTIFTNYAFEWNLPSYTLEAVAEALLGXXKEKXDXXVYAEEIAXAWNEXGENLERLARYSXEDAELTXELGAKKEFLPXEXLIQLSRVXGQPLDWDVSRSSTGNLVEWLLLRKAYERNEVAPNKPSEXEXARRXXXRESALIKXXXYPGGYVWLNDRFKEPEXGLYEXNIVXLDFRSLYPSIIXTHNLSPDTLLNREGCXPYHXYIXDVAPEEXXVXXXSGHXFCXTKDFPGFIPSILIKRGDLLEERKKIKKSGGKMXAXXXNXEXIKXXXXXXXLSXXEXXXVXXXXXFSDXFKKTIDPNTXXIEKKLLDYRQRAIXXXXTXQXNRKILANSFYGYLGYAKARWYCXECAESVTAXGFRQYIEXTIREIEEKFGKVCGTNXEAFXXXXAXXCXXNGYXXGFKVLYGDTDSXFVTIPGXIXKVGXXXFADAEEXKKXILXFKXXEFLKYINAWWREXXXYMNKLPGLLELEYEGFYKRGLFXXGRGXFVGTKKRYAGLIDEMXXDRFAEGKITTKGLETVRRDWSELAKETQARVLEXILKXEPEGDVEEAEESVVXYVKEVIEKLXXYEVPPXAGXIEKLVIHKQLTRDLDDYKATGFPGXKCPXHVAVAKRLDRANAARGRGVKXRPGTVISYVILKGGGXIGDXDRAIPPDEXDPXKHKYDAEYYIENQVLXXPAVERILEAFIGDAFAXXXXXRGDHXXXXXXXTGYRKEDLXXFXKXXXXCXXCXXXLXXGXGXXCXXCXXRYQKTKQVGLXAWXXLKPKGKRLWTQCQRCXGNLHXXVXCXXXXCXIFYMRXKVXKXLXDXXXXLXXFGPPGPFXF

SEQ ID NO: 2 - protein, 773 aa, Thermococcus gorgonarius
MILDTDYITEDGKPVIRIFKKENGEFKIDYDRNFEPYIYALLKDDSAIEDVKKITAERHGTTVRVVRAEKVKKKFLGRPIEVWKLYFTHPQDVPAIRDKIKEHPAVVDIYEYDIPFAKRYLIDKGLIPMEGDEELKMLAFDIETLYHEGEEFAEGPILMISYADEEGARVITWKNIDLPYVDVVSTEKEMIKRFLKVVKEKDPDVLITYNGDNFDFAYLKKRSEKLGVKFILGREGSEPKIQRMGDRFAVEVKGRIHFDLYPVIRRTINLPTYTLEAVYEAIFGQPKEKVYAEEIAQAWETGEGLERVARYSMEDAKVTYELGKEFFPMEAQLSRLVGQSLWDVSRSSTGNLVEWFLLRKAYERNELAPNKPDERELARRRESYAGGYVKEPERGLWENIVYLDFRSLYPSIIITHNVSPDTLNREGCEEYDVAPQVGHKFCKDFPGFIPSLLGDLLEERQKVKKKMKATIDPIEKKLLDYRQRAIKILANSFYGYYGYAKARWYCKECAESVTAWGRQYIETTIREIEEKFGFKVLYADTDGFFATIPGADAETVKKKAKEFLDYINAKLPGLLELEYEGFYKRGFFVTKKKYAVIDEEDKITTRGLEIVRRDWSEIAKETQARVLEAILKHGDVEEAVRIVKEVTEKLSKYEVPPEKLVIYEQITRDLKDYKATGPHVAVAKRLAARGIKIRPGTVISYIVLKGSGRIGDRAIPFDEFDPAKHKYDAEYYIENQVLPAVERILRAFGYRKEDLRYQKTRQVGLGAWLKPKT

SEQ ID NO: 3 - protein, 774 aa, Thermococcus kodakarensis
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFDIETLYEEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRAIKILANSYYGYYGYARARWYCKECAESVTAWGREYITMTIKEIEEKYGFKVIYSDTDGFFATIPGADAETVKKKAMEFLKYINAKLPGALELEYEGFYERGFFVTKKKYAVIDEEGKITTRGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 4 - protein, 775 aa, Thermococcus sp.
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 5 - protein, 775 aa, Pyrococcus furiosus
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 6 - protein, 774 aa, Thermococcus litoralis
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRGKIREHPAVVDIYEYDIPFAKRYLIDKGLIPMEGDEELKLLAFDIETFYHEGDEFGKGEIIMISYADEEEARVITWKNIDLPYVDVVSNEREMIKRFVQVVKEKDPDVIITYNGDNFDLPYLIKRAEKLGVRLVLGRDKEHPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLYPSIIVTHNVSPDTLEKEGCKNYDVAPIVGYRFCKDFPGFIPSILGDLIAMRQDIKKKMKSTIDPIEKKMLDYRQRAIKLLANSYYGYMGYPKARWYSKECAESVTAWGRHYIEMTIREIEEKFGFKVLYADTDGFYATIPGEKPELIKKKAKEFLNYINSKLPGLLELEYEGFYLRGFFVTKKRYAVIDEEGRITTRGLEVVRRDWSEIAKETQAKVLEAILKEGSVEKAVEVVRDVVEKIAKYRVPLEKLVIHEQITRDLKDYKAIGPHVAIAKRLAARGIKVKPGTIISYIVLKGSGKISDRVILLTEYDPRKHKYDPDYYIENQVLPAVLRILEAFGYRKEDLRYQSSKQTGLDAWLKR

SEQ ID NO: 7 - protein, 937 aa, Methanosarcina acetivorans
MPMDFQILDADYEVVNDSGPVIRLFGRGADGKSVCCFVPDFEPYFYLKASGDLHAVARLIKDTFEQVKKVEIVEKFEPVGYQKTKKEMLRVTTRLPKDVPEIRDEILKIRDVLRAEGDWQVYESDILFRNRFLIDRALGGMVWVSAEGKPVDPVRYLGAGSAWRSRCENFACDSAVLASGLKRVENLAIAPLKYLAFDIECLPLDGGMPSPDVSPIIMISFSFEPEYKGHKTLILLAKPAAGMDGDVLSCMDETEMLNKFFEIICEYDPDIVAGYNHQDFDIPYITERVKALVAKGETINSVVGRDGSPIGYRKFGLITRTEMKGRVVVDALPLVRRAFSLKQYTLRAVSKELLSREKLDVPPLEMEEHWNDSGDKFRKFVDYARRDSELALELVLELRLLDKYIALAQVSGSLLQEIVDGGQTSMVETLLLREFGLKDRVILPKPGDELSAERYDMSSDLKGGEVLEPKKGLLENVLILDYKSLYPTIMMAHNLCYTTVVTRDRPDGKTIKPPSGGEFVPPEVFRGIVPSILEDLLNKRGDTKKRMKRTSDENEHRVLDATQLAIKILLNSFYGYSGYARARLYSLTLANAVTSFGRSNILNTRDLINGRIGKIVLRNSAALLLEEAGKLSPQDRIVELSVAYGDTDSVFVHCKAKGDLSLEEVSLVGNRLSEIVSASLPDPMELEFESVAKRALLIAKKRYALWLFEPRNSGWENKIKVKGMETVRRDWCELTSITLNRVLEFVLIEGDVDKAVEHVRKVVSDVRNLDPGKDAGIIEKLVLTRTLTRKADSYKNKQPHLTVAENLKKRTGIMPSIGTRIPFVITAGKGLFVDRAEDPDYVRENNVPIDVDYYVKKQILPPVERILEVFGVKMSSLDFDAKQKGLFDFEVKKPEAKKQEKSSSQKGTNGKILEKAPEEKARYSENGRVEQRSLFDF

SEQ ID NO: 8 - protein, 785 aa, Pyrobaculum islandicum
MELKVWPLDITYAVVGSVPEIRIFGILSSGERVVLIDRSFKPYFYVDCAVCEPAALKTALSRVAPIDDVQIVERRFLGRSKKFLKVIAKIPEDVRKLREAAMSIPRVSGVYEADIRFYMRYMIDMGVVPCSWNVAEVEEGGRLGGIPTYVVSQWYGIDEGFPPSLKVMAFDIEVYNERGSPDPIRDPVVMLAIKTNDGHEEVFEASGKDDRGVVRAFVDFIRSYDPDVIVGYNSNGFDWPYLVERAKAVGVPLKVDRLSNPPQQSVYGHWSIVGRANVDLYNIVEEFPEIKLKTLDRVAEYFGVMKREERVLIPGHKIYEYWKDPNKRPLLKRYVLDDVRSTLGLADKLLPFLIQLSSVSGLPLDQVAAASVGNRVEWMLLRYAYRLGEVAPNREEREYEPYKGAIVLEPKPGMYEDVLVLDFSSMYPNIMMKYNLSPDTYLEPGEPDPPEGVNVAPEVGHRFRRSPPGFVPQVLKSLVELRKAVREEAKKYPPDSPEFKILDERQRALKVMANAIYGYLGWVGARWYKREVAESVTAFARAILKDVIEQARRLGIVVVYGDTDSLFVKKHGDVDKLIKYVEEKYGIDIKVDKDYAKVLFTEAKKRYAGLLRDGRIDIVGFEVVRGDWSELAKDVQLRVIEIILKSRDIVEARHGVIKYIREIIERLKNYKFNIDDLIIWKTLDKELDEYKAYPPHVHAAQILKRHGYRVGKGTTIGYVIVKGGEKVSERALPYILLDDIKKIDIDYYIERQIIPAALRIAEVIGVKESDLKTGRMERSLLDFLS

SEQ ID NO: 9 - protein, 882 aa, Sulfolobus solfataricus
MTKQLTLFDIPSSKPAKSEQNTQQSQQSAPVEEKKVVRREWLEEAQENKIYFLLQVDYDGKKGKAVCKLFDKETQKIYALYDNTGHKPYFLVDLEPDKVGKIPKIVRDPSFDHIETVSKIDPYTWNKFKLTKIVVRDPLAVRRLRNDVPKAYEAHIKYFNNYMYDIGLIPGMPYVVKNGKLESVYLSLDEKDVEEIKKAFADSDEMTRQMAVDWLPIFETEIPKIKRVAIDIEVYTPVKGRIPDSQKAEFPIISIALAGSDGLKKVLVLNRNDVNEGSVKLDGISVERFNTEYELLGRFFDILLEYPIVLTFNGDDFDLPYIYFRALKLGYFPEEIPIDVAGKDEAKYLAGLHIDLYKFFFNKAVRNYAFEGKYNEYNLDAVAKALLGTSKVKVDTLISFLDVEKLIEYNFRDAEITLQLTTFNNDLTMKLIVLFSRISRLGIEELTRTEISTWVKNLYYWEHRKRNWLIPLKEEILAKSSNIRTSALIKGKGYKGAVVIDPPAGIFFNITVLDFASLYPSIIRTWNLSYETVDIQQCKKPYEVKDETGEVLHIVCMDRPGITAVITGLLRDFRVKIYKKKAKNPNNSEEQKLLYDVVQRAMKVFINATYGVFGAETFPLYAPAVAESVTALGRYVITSTVKKAREEGLTVLYGDTDSLFLLNPPKNSLENIIKWVKTTFNLDLEVDKTYKFVAFSGLKKNYFGVYQDGKVDIKGMLVKKRNTPEFVKKVFNEVKELMISINSPNDVKEIKRKIVDVVKGSYEKLKNKGYNLDELAFKVMLSKPLDAYKKNTPQHVKAALQLRPFGVNVLPRDIIYYVKVRSKDGVKPVQLAKVTEIDAEKYLEALRSTFEQILRAFGVSWDEIAATMSIDSFFSYPSKGNS

SEQ ID NO: 10 - protein, 784 aa, Methanococcus maripaludis
MESLIDLDYNSDDLCIYLYLINSIIKEKDFKPYFYVNSTDKEQILEFLKDYEKKHKLDSEISKMIENIETVKKIVFDENYQEKELSKVTVKYPNNVKTVREILMEFERLYEYDIPFVRRYLIDNSVIPTSTWDFENNKKIDNKIPDFKTVSFDIEVYCNKEPNPKKDPIIMASFSSKDFNTVVSTKKFDHEKLEYVKDEKELIKRIIEILKEYDIIYTYNGDNFDFPYLKKRAESFGLELKLGKNDEKIKITKGGMNSKSYIPGRVHIDLYPIARRLLNLTKYRLENVTEALFDVKKVDVGHENIPKMWDNLDETLVEYSHQDAYYTQRIGEQFLPLEIMFSRVVNQSLYDINRMSSSQMVEYLLLKNSYKMGVIAPNRPSGKEYQKRIRSSYEGGYVKEPLKGIHEDIVSMDFLSLYPSIIMSHNLSPETIDCTCCSDEENGENEEILGHKFCKKSIGIIPKTLMDLINRRKKVKKVLREKAEKGEFDEEYQILDYEQRSIKVLANSHYGYLAFPMARWYSRDCAEITTHLGRQYIQKTIEEAENFGFKVIYADTDGFYSKWADDKEKLSKYELLEKTREFLKNINNTLPGEMELEFEGYFKRGIFVTKKKYALIDENEKITVKGLEVVRRDWSNVSKNTQKNVLNALLKEGSVENAKKVIQDTIKELKDGKVNNEDLLIHTQLTKRIEDYKTTAPHVEVAKKILKSGNRVNTGDVISYIITSGNKSISERAEILENAKNYDTNYYIENQILPPVIRLMEALGITKDELKDSKKQYTLHHFLK

SEQ ID NO: 11 - protein, 1107 aa, Homo sapiens
MDGKRRPGPGPGVPPKRARGGLWDDDDAPRPSQFEEDLALMEEMEAEHRLQEQEEEELQSVLEGVADGQVPPSAIDPRWLRPTPPALDPQTEPLIFQQLEIDHYVGPAQPVPGGPPPSRGSVPVLRAFGVTDEGFSVCCHIHGFAPYFYTPAPPGFGPEHMGDLQRELNLAISRDSRGGRELTGPAVLAVELCSRESMFGYHGHGPSPFLRITVALPRLVAPARRLLEQGIRVAGLGTPSFAPYEANVDFEIRFMVDTDIVGCNWLELPAGKYALRLKEKATQCQLEADVLWSDVVSHPPEGPWQRIAPLRVLSFDIECAGRKGIFPEPERDPVIQICSLGLRWGEPEPFLRLALTLRPCAPILGAKVQSYEKEEDLLQAWSTFIRIMDPDVITGYNIQNFDLPYLISRAQTLKVQTFPFLGRVAGLCSNIRDSSFQSKQTGRRDTKVVSMVGRVQMDMLQVLLREYKLRSYTLNAVSFHFLGEQKEDVQHSIITDLQNGNDQTRRRLAVYCLKDAYLPLRLLERLMVLVNAVEMARVTGVPLSYLLSRGQQVKVVSQLLRQAMHEGLLMPVVKSEGGEDYTGATVIEPLKGYYDVPIATLDFSSLYPSIMMAHNLCYTTLLRPGTAQKLGLTEDQFIRTPTGDEFVKTSVRKGLLPQILENLLSARKRAKAELAKETDPLRRQVLDGRQLALKVSANSVYGFTGAQVGKLPCLEISQSVTGFGRQMIEKTKQLVESKYTVENGYSTSAKVVYGDTDSVMCRFGVSSVAEAMALGREAADWVSGHFPSPIRLEFEKVYFPYLLISKKRYAGLLFSSRPDAHDRMDCKGLEAVRRDNCPLVANLVTASLRRLLIDRDPEGAVAHAQDVISDLLCNRIDISQLVITKELTRAASDYAGKQAHVELAERMRKRDPGSAPSLGDRVPYVIISAAKGVAAYMKSEDPLFVLEHSLPIDTQYYLEQQLAKPLLRIFEPILGEGRAEAVLLRGDHTRCKTVLTGKVGGLLAFAKRRNCCIGCRTVLSHQGAVCEFCQPRESELYQKEVSHLNALEERFSRLWTQCQRCQGSLHEDVICTSRDCPIFYMRKKVRKDLEDQEQLLRRFGPPGPEAW

SEQ ID NO: 12 - protein, 1097 aa, Saccharomyces cerevisiae
MSEKRSLPMVDVKIDDEDTPQLEKKIKRQSIDHGVGSEPVSTIEIIPSDSFRKYNSQGFKAKDTDLMGTQLESTFEQELSQMEHDMADQEEHDLSSFERKKLPTDFDPSLYDISFQQIDAEQSVLNGIKDENTSTVVRFFGVTSEGHSVLCNVTGFKNYLYVPAPNSSDANDQEQINKFVHYLNETFDHAIDSIEVVSKQSIWGYSGDTKLPFWKIYVTYPHMVNKLRTAFERGHLSFNSWFSNGTTTYDNIAYTLRLMVDCGIVGMSWITLPKGKYSMIEPNNRVSSCQLEVSINYRNLIAHPAEGDWSHTAPLRIMSFDIECAGRIGVFPEPEYDPVIQIANVVSIAGAKKPFIRNVFTLNTCSPITGSMIFSHATEEEMLSNWRNFIIKVDPDVIIGYNTTNFDIPYLLNRAKALKVNDFPYFGRLKTVKQEIKESVFSSKAYGTRETKNVNIDGRLQLDLLQFIQREYKLRSYTLNAVSAHFLGEQKEDVHYSIISDLQNGDSETRRRLAVYCLKDAYLPLRLMEKLMALVNYTEMARVTGVPFSYLLARGQQIKVVSQLFRKCLEIDTVIPNMQSQASDDQYEGATVIEPIRGYYDVPIATLDFNSLYPSIMMAHNLCYTTLCNKATVERLNLKIDEDYVITPNGDYFVTTKRRRGILPIILDELISARKRAKKDLRDEKDPFKRDVLNGRQLALKISANSVYGFTGATVGKLPCLAISSSVTAYGRTMILKTKTAVQEKYCIKNGYKHDAVVVYGDTDSVMVKFGTTDLKEAMDLGTEAAKYVSTLFKHPINLEFEKAYFPYLLINKKRYAGLFWTNPDKFDKLDQKGLASVRRDSCSLVSIVMNKVLKKILIERNVDGALAFVRETINDILHNRVDISKLIISKTLAPNYTNPQPHAVLAERMKRREGVGPNVGDRVDYVIIGGNDKLYNRAEDPLFVLENNIQVDSRYYLTNQLQNPIISIVAPIIGDKQANGMFVVKSIKINTGSQKGGLMSFIKKVEACKSCKGPLRKGEGPLCSNCLARSGELYIKALYDVRDLEEKYSRLWTQCQRCAGNLHSEVLCSNKNCDIFYMRVKVKKELQEKVEQLSKW

SEQ ID NO: 13 - protein, 787 aa, Pseudomonas aeruginosa
MELLQGFVLTRHWRDTPAGTEVAFWLATEQGPRQVRLPPQPSVAFVLAEQRGRVESLLAGETGAELRPLALRDFQQRPVLGLYCQQHRQLMNLEKRLRQAGVEVFEADIRPPERYLMERFITAPVSLEASVEADGSLLARRLKPAPDYRPRLRLVSLDIETNARGDLYSIALEGCDQRQVYMLGPANGDAAAVDFRLDYCDSRAGLLERLNQWLAEHDPDAIIGWNLVQFDLRVLHEHAQRLKVPLRLGRGGDEMGWREHGSRNNHFFAAAAGRLIIDGIEALRSATWSFPSFSLENVARTLLGEGKAIDNPYQRMDEIDRMFAEDKPALAHYNLKDCELVTRIFARTELLDFLLERATVTGLPADRSGGSVAAFTHLYMPLMHRAGFVAPNLGEKRPEASPGGFVMDSRPGLYESVLVLDYKSLYPSIIRTFLIDPVGLVEGLRQPDDEHSVEGFRGARFSRTRHCLPAIVARVWEGREAAKRERNQPLSQALKIIMNAFYGVLGSSGCRFFDPRLASSITLRGHRIMRRTRELIEAEGYTVIYGDTDSTFVWLGSPRAEEEAAAIGRALVARVNDWWREHLKEEFGLDSALELQFETHYRRFLMPTVRGAEEGSKKRYAGLVRRADGGEEMVFKGLETVRTDWSPLAQRFQQELYLRIFNRQPYQDYVRDYVRRTLAGELDDLLVYRKRLRRRLDDYQRNVPPHVRAARIADDYNLERGRPRQYQSGGWISYVISVAGPEPLEARRSAIDYEHYVGKQLQPVADAILPFVGDDFATLVDRQMALF

SEQ ID NO: 14 - protein, 783 aa, Escherichia coli
MAQAGFILTRHWRDTPQGTEVSFWLATDNGPLQVTLAPQESVAFIPADQVPRAQHILQGEQGFRLTPLALKDFHRQPVYGLYCRAHRQLMNYEKRLREGGVTVYEADVRPPERYLMERFITSPVWVEGDMHNGTIVNARLKPHPDYRPPLKWVSIDIETTRHGELYCIGLEGCGQRIVYMLGPENGDASSLDFELEYVASRPQLLEKLNAWFANYDPDVIIGWNVVQFDLRMLQKHAERYRLPLRLGRDNSELEWREHGFKNGVFFAQAKGRLIIDGIEALKSAFWNFSSFSLETVAQELLGEGKSIDNPWDRMDEIDRRFAEDKPALATYNLKDCELVTQIFHKTEIMPFLLERATVNGLPVDRHGGSVAAFGHLYFPRMHRAGYVAPNLGEVPPHASPGGYVMDSRPGLYDSVLVLDYKSLYPSIIRTFLIDPVGLVEGMAQPDPEHSTEGFLDAWFSREKHCLPEIVTNIWHGRDEAKRQGNKPLSQALKIIMNAFYGVLGTTACRFFDPRLASSITMRGHQIMRQTKALIEAQGYDVIYGDTDSTFVWLKGAHSEEEAAKIGRALVQHVNAWWAETLQKQRLTSALELEYETHFCRFLMPTIRGADTGSKKRYAGLIQEGDKQRMVFKGLETVRTDWTPLAQQFQQELYLRIFRNEPYQEYVRETIDKLMAGELDARLVYRKRLRRPLSEYQRNVPPHVRAARLADEENQKRGRPLQYQNRGTIKYVWTTNGPEPLDYQRSPLDYEHYLTRQLQPVAEGILPFIEDNFATLMTGQLGLF

SEQ ID NO: 15 - protein, 903 aa, Escherichia coli phage
MKEFYLTVEQIGDSIFERYIDSNGRERTREVEYKPSLFAHCPESQATKYFDIYGKPCTRKLFANMRDASQWIKRMEDIGLEALGMDDFKLAYLSDTYNYEIKYDHTKIRVANFDIEVTSPDGFPEPSQAKHPIDAITHYDSIDDRFYVFDLLNSPYGNVEEWSIEIAAKLQEQGGDEVPSEIIDKIIYMPFDNEKELLMEYLNFWQQKTPVILTGWNVESFDIPYVYNRIKNIFGESTAKRLSPHRKTRVKVIENMYGSREIITLFGISVLDYIDLYKKFSFTNQPSYSLDYISEFELNVGKLKYDGPISKLRESNHQRYISYNIIDVYRVLQIDAKRQFINLSLDMGYYAKIQIQSVFSPIKTWDAIIFNSLKEQNKVIPQGRSHPVQPYPGAFVKEPIPNRYKYVMSFDLTSLYPSIIRQVNISPETIAGTFKVAPLHDYINAVAERPSDVYSCSPNGMMYYKDRDGVVPTEITKVFNQRKEHKGYMLAAQRNGEIIKEALHNPNLSVDEPLDVDYRFDFSDEIKEKIKKLSAKSLNEMLFRAQRTEVAGMTAQINRKLLINSLYGALGNVWFRYYDLRNATAITTFGQMALQWIERKVNEYLNEVCGTEGEAFVLYGDTDSIYVSADKIIDKVGESKFRDTNHWVDFLDKFARERMEPAIDRGFREMCEYMNNKQHLMFMDREAIAGPPLGSKGIGGFWTGKKRYALNVWDMEGTRYAEPKLKIMGLETQKSSTPKAVQKALKECIRRMLQEGEESLQEYFKEFEKEFRQLNYISIASVSSANNIAKYDVGGFPGPKCPFHIRGILTYNRAIKGNIDAPQVVEGEKVYVLPLREGNPFGDKCIAWPSGTEITDLIKDDVLHWMDYTVLLEKTFIKPLEGFTSAAKLDYEKKASLFDMFDF

SEQ ID NO: 16 - protein, 898 aa, Escherichia coli phage
MKEFYISIETVGNNIVERYIDENGKERTREVEYLPTMFRHCKEESKYKDIYGKNCAPQKFPSMKDARDWMKRMEDIGLEALGMNDFKLAYISDTYGSEIVYDRKFVRVANCDIEVTGDKFPDPMKAEYEIDAITHYDSIDDRFYVFDLLNSMYGSVSKWDAKLAAKLDCEGGDEVPQEILDRVIYMPFDNERDMLMEYINLWEQKRPAIFTGWNIEGFDVPYIMNRVKMILGERSMKRFSPIGRVKSKLIQNMYGSKEIYSIDGVSILDYLDLYKKFAFTNLPSFSLESVAQHETKKGKLPYDGPINKLRETNHQRYISYNIIDVESVQAIDKIRGFIDLVLSMSYYAKMPFSGVMSPIKTWDAIIFNSLKGEHKVIPQQGSHVKQSFPGAFVFEPKPIARRYIMSFDLTSLYPSIIRQVNISPETIRGQFKVHPIHEYIAGTAPKPSDEYSCSPNGWMYDKHQEGIIPKEIAKVFFQRKDWKKKMFAEEMNAEAIKKIIMKGAGSCSTKPEVERYVKFSDDFLNELSNYTESVLNSLIEECEKAATLANTNQLNRKILINSLYGALGNIHFRYYDLRNATAITIFGQVGIQWIARKINEYLNKVCGTNDEDFIAAGDTDSVYVCVDKVIEKVGLDRFKEQNDLVEFMNQFGKKKMEPMIDVAYRELCDYMNNREHLMHMDREAISCPPLGSKGVGGFWKAKKRYALNVYDMEDKRFAEPHLKIMGMETQQSSTPKAVQEALEESIRRILQEGEESVQEYYKNFEKEYRQLDYKVIAEVKTANDIAKYDDKGWPGFKCPFHIRGVLTYRRAVSGLGVAPILDGNKVMVLPLREGNPFGDKCIAWPSGTELPKEIRSDVLSWIDHSTLFQKSFVKPLAGMCESAGMDYEEKASLDFLFG

SEQ ID NO: 17 - protein, 575 aa, Bacillus phage [sequence omitted]

SEQ ID NO: 18 - DNA, 45 bp, synthetic construct
  misc_feature 1..45: note = FAM-45-mer DNA initiator
  source 1..45: mol_type = other DNA
ctcggcctggcacaggtccgttcagtgctgcggcgaccaccgagg

(19) United States
(12) Patent Application Publication
(10) Pub. No.: US 2025/0257335 A1
(43) Pub. Date: Aug. 14, 2025

(54) NUCLEIC ACID POLYMERASE VARIANTS, KITS AND METHODS FOR TEMPLATE-INDEPENDENT RNA SYNTHESIS

(71) Applicant: CHENG-YAO CHEN, Hsinchu County (TW)

(72) Inventors: Cheng-Yao Chen, Hsinchu County (TW); Yi-Wen Cheng, Hsinchu County (TW); Yu Ting Hung, Hsinchu County (TW); Wen-Ting Chen, Hsinchu County (TW)

(73) Assignee: Cheng-Yao Chen, Hsinchu County (TW)

(21) Appl. No.: 19/060,810

(22) Filed: Feb. 24, 2025

Related U.S. Application Data
(63) Continuation of application No. 17/936,744, filed on Sep. 29, 2022.
(60) Provisional application No. 63/249,819, filed on Sep. 29, 2021.

(51) Int. Cl. C12N 9/12; C12N 15/09

(52) U.S. Cl. CPC C12N 9/1252; C12N 9/1247; C12N 15/09; C12Y 207/07007

(57) ABSTRACT

Provided herein relates to nucleic acid polymerase variants and kits including the same, where the nucleic acid polymerase variant has an improved function and activity of performing template-independent nucleic acids synthesis using ribonucleotides (rNTPs) in a thermotolerant manner.

### CROSS REFERENCE

This application is a continuation application of U.S. application Ser. No. 17/936,744, filed on Sep. 29, 2022, which claims the benefit of U.S. Provisional Application No. 63/249,819, filed on Sep. 29, 2021. The contents of these applications are incorporated herein by reference.

### TECHNICAL FIELD

The present disclosure relates to nucleic acid polymerase variants and kits comprising the same for use particularly in the context of de novo enzymatic nucleic acid synthesis.

### SEQUENCE LISTING

The present application contains a computer readable Sequence Listing which has been submitted electronically in XML format and is hereby incorporated by reference in its entirety. Said XML format file, created on May 2, 2025, is named YDBP0012USA3_A3_1Sequence Listing XML.xml and is 92.9 kilobytes in size.

### BACKGROUND

Synthetic oligonucleotides are crucial to many aspects of biotechnological research in both the academic and industrial settings. However, there are many limitations of traditional chemical synthesis methods developed decades ago. This is especially true for de novo RNA synthesis, which remains largely inaccessible to those heavily invested in advancing genome engineering technologies, RNA-based diagnostics, RNA-based therapeutics, RNA-based sequencing technologies, nucleic acid-based information storage, and even biological computing. Chemical synthesis of RNA is troubled by lengthy reaction steps that require both harsh chemical reagents and biologically incompatible organic solvents. These reaction conditions often lead to the depurination of the nucleotide bases, unexpected insertions or deletions from the overall sequence, and the premature irreversible capping of the oligonucleotide resulting in unwanted truncated products. This substantially increases the overall error-rate of RNA synthesis, limits the synthesis length of RNA oligonucleotides (less than 120 nucleotides), and requires longer lead-times to obtain acceptable yields of a desired product. Moreover, the chemical synthesis of RNA oligonucleotide is toxic and labor-intensive. Overcome the current limitations of RNA oligonucleotide synthesis is therefore important.

Enzyme-based de novo nucleic acid synthesis is an emerging, non-toxic method to substitute for the decades-old, toxic, chemical phosphoramidite-based nucleic acid synthesis. All living organisms rely on nucleic acid polymerases to efficiently duplicate their nucleic acid. Owing to their nucleic acid duplication mechanism, most nucleic acid polymerases require a template to direct synthesis and incorporation of nucleotides into a growing nucleic acid strand. The template-dependent manner of nucleic acid synthesis requires the polymerase to associate with a primer-template nucleic acid before the nucleotide can be added to the 3′-terminus of primer by the polymerase.

Unlike most replicative nucleic acid polymerases, the X-family terminal deoxynucleotidyl transferase (Tdt) is a unique class of mesophilic enzyme, which doesn't rely on a template for adding nucleotides during nucleic acid synthesis. Tdt only requires a short initiator DNA or primer to direct synthesis and incorporation of nucleotides into a growing initiator DNA or primer. Previous studies revealed that Tdt can incorporate both deoxyribonucleotides (dNTPs) and ribonucleotides (rNTPs) with a minor discrimination (Bould, J B et al. The Journal of biological chemistry (2001)). However, Tdt fails to extend the newly synthesized RNA strand beyond around 4-5 ribonucleotides, suggesting that Tdt has impeded accommodation of ribo- or mixed ribo/deoxyribonucleic acid substrates for further synthesis. Therefore, Tdt enzyme is not suitable for de novo RNA synthesis.

### SUMMARY OF THE INVENTION

Owing to the diverse structure-function relationships mentioned above, the naturally occurring, replicative nucleic acid polymerases cannot readily utilize ribonucleotides (rNTPs) as a substrate for de novo ribonucleic acid synthesis. Thus, the tailor-made, modified nucleic acid polymerase is a prerequisite for exerting the utilities of a variety of nucleic acid synthesis applications.

The inventor has discovered the novel positions/regions in the amino acid sequences of B-family DNA polymerase variants that play crucial parts in endowing the said polymerases with a template-independence and an enhancing nucleotide substrate binding affinity of said polymerases for ribonucleotides, thereby providing a new option for the template-independent RNA synthesis method.

Accordingly, in one aspect, the present disclosure provides a, so-called, RNA polymerase variant comprising: a motif A, and a motif B corresponding to positions 706 to 730, and 843 to 855, respectively, of a consensus sequence (SEQ ID NO:1); and at least one amino acid substitution at a position in the motif A, the motif B, or the combination thereof; wherein the RNA polymerase variant has a reduced or eliminated the intrinsic 3′ to 5′ exonuclease activity.

In one embodiment, the representative RNA polymerase variant is modified from a wild-type B-family DNA polymerase having an amino acid sequence selected from the group consisting of SEQ ID NO: 2, 3, 4, 5, 6, 7, 8, 9, 10, 11, 12, 13, 14, 15, 16 and 17.

In one embodiment, the wild-type B-family DNA polymerase is Thermococcus gorgonarius DNA polymerase (Tgo), Thermococcus kodakarensis DNA polymerase (Kod1), Thermococcus sp. (strain 9° N-7) DNA polymerase (9° N), Pyrococcus furiosus DNA polymerase (Pfu), Thermococcus litoralis DNA polymerase (Vent), Methanosarcina acetivorans DNA polymerase (Mac), Pyrobaculum islandicum DNA polymerase (Pis), Sulfolobus solfataricus DNA polymerase (Sso), Methanococcus maripaludis DNA polymerase (Mma), human DNA polymerase, delta catalytic p125 subunit (hPOLD), Saccharomyces cerevisiae DNA polymerase delta catalytic subunit (ScePOLD), Pseudomonas aeruginosa DNA polymerase II (Pae), Escherichia. coli DNA polymerase II (Eco), Escherichia phage RB69 DNA polymerase (RB69), Escherichia phage T4 DNA polymerase (T4), or Bacillus phage Phi29 DNA polymerase (Phi29).

In one embodiment, the representative RNA polymerase variant comprises a motif Exo I corresponding to positions 349 to 364 of the consensus sequence (SEQ ID NO:1), and the RNA polymerase variant has at least one amino acid substitution at a position in the motif Exo I. Preferably, an amino acid L or M corresponding to position 715 of SEQ ID NO: 1 is substituted with A, C, D, F, G, H, K, N, Q, S, W, or Y; an amino acid Y corresponding to position 716 of SEQ ID NO: 1 remains unchanged or is substituted with A, C, D, G, N, S, T or V; and an amino acid P corresponding to position 717 of SEQ ID NO: 1 remains unchanged or is substituted with A, C, G, I, L, M, N, S, T or V.

In one embodiment, the selected RNA polymerase variant is derived from Thermococcus gorgonarius DNA polymerase (Tgo) having a wild-type amino acid sequence of SEQ ID NO: 2; and wherein: an amino acid L at position 408 of SEQ ID NO: 2 is substituted with A, C, D, F, G, H, K, M, N, Q, S, W, or Y; an amino acid Y at position 409 of SEQ ID NO: 2 remains unchanged or is substituted with A, C, D, G, N, S, T or V; and an amino acid P at position 410 of SEQ ID NO: 2 remains unchanged or is substituted with A, C, G, I, L, M, N, S, T or V.

In one embodiment, the representative RNA polymerase variant is derived from Thermococcus gorgonarius DNA polymerase (Tgo) having a wild-type amino acid sequence of SEQ ID NO: 2; and wherein: an amino acid L at position 408 of SEQ ID NO: 2 is substituted with A, C, D, F, G, H, K, M, N, Q, S, W, or Y; an amino acid Y at position 409 of SEQ ID NO: 2 remains unchanged or is substituted with A, C, D, G, N, S, T or V; an amino acid P at position 410 of SEQ ID NO: 2 remains unchanged or is substituted with A, C, G, I, L, M, N, S, T or V; and an amino acid A at position 485 of SEQ ID NO: 2 is substituted with C, D, E, F, G, H, I, K, L, M, N, P, Q, R, T, V, W or Y.

In one embodiment, the representative RNA polymerase variant is derived from Thermococcus kodakarensis DNA polymerase (Kod1) having a wild-type amino acid sequence of SEQ ID NO: 3; and wherein: an amino acid L at position 408 of SEQ ID NO: 3 is substituted with A, C, D, F, G, H, K, M, N, Q, S, W, or Y; an amino acid Y at position 409 of SEQ ID NO: 3 remains unchanged or is substituted with A, C, D, G, N, S, T or V; and an amino acid P at position 410 of SEQ ID NO: 3 remains unchanged or is substituted with A, C, G, I, L, M, N, S, T or V.

In one embodiment, the representative RNA polymerase variant is derived from Thermococcus kodakarensis DNA polymerase (Kod1) having a wild-type amino acid sequence of SEQ ID NO: 3; and wherein: an amino acid L at position 408 of SEQ ID NO: 3 is substituted with A, C, D, F, G, H, K, M, N, Q, S, W, or Y; an amino acid Y at position 409 of SEQ ID NO: 3 remains unchanged or is substituted with A, C, D, G, N, S, T or V; an amino acid P at position 410 of SEQ ID NO: 3 remains unchanged or is substituted with A, C, G, I, L, M, N, S, T or V; and an amino acid A at position 485 of SEQ ID NO: 3 is substituted with C, D, E, F, G, H, I, K, L, M, N, P, Q, R, T, V, W or Y.

In one embodiment, the representative RNA polymerase variant is derived from Thermococcus sp. (strain 9° N-7) DNA polymerase (9° N) having a wild-type amino acid sequence of SEQ ID NO: 4; and wherein: an amino acid L at position 408 of SEQ ID NO: 4 is substituted with A, C, D, F, G, H, K, M, N, Q, S, W, or Y; an amino acid Y at position 409 of SEQ ID NO: 4 is remains unchanged or is substituted with A, C, D, G, N, S, T or V; and an amino acid P at position 410 of SEQ ID NO: 4 remains unchanged or is substituted with A, C, G, I, L, M, N, S, T or V.

In one embodiment, the representative RNA polymerase variant is derived from Thermococcus sp. (strain 9° N-7) DNA polymerase (9° N) having a wild-type amino acid sequence of SEQ ID NO: 4; and wherein: an amino acid L at position 408 of SEQ ID NO: 4 is substituted with A, C, D, F, G, H, K, M, N, Q, S, W, or Y; an amino acid Y at position 409 of SEQ ID NO: 4 remains unchanged or is substituted with A, C, D, G, N, S, T or V; an amino acid P at position 410 of SEQ ID NO: 4 remains unchanged or is substituted with A, C, G, I, L, M, N, S, T or V; and an amino acid A at position 485 of SEQ ID NO: 4 is substituted with C, D, E, F, G, H, I, K, L, M, N, P, Q, R, T, V, W or Y.

In one embodiment, the representative RNA polymerase variant is derived from Pyrococcus furiosus DNA polymerase (Pfu) having a wild-type amino acid sequence of SEQ ID NO: 5; and wherein: an amino acid L at position 409 of SEQ ID NO: 5 is substituted with A, C, D, F, G, H, K, M, N, Q, S, W, or Y; an amino acid Y at position 410 of SEQ ID NO: 5 remains unchanged or is substituted with A, C, D, G, N, S, T or V; and an amino acid P at position 411 of SEQ ID NO: 5 remains unchanged or is substituted with A, C, G, I, L, M, N, S, T or V.

In one embodiment, the representative RNA polymerase variant is derived from Pyrococcus furiosus DNA polymerase (Pfu) having a wild-type amino acid sequence of SEQ ID NO: 5; and wherein: an amino acid L at position 409 of SEQ ID NO: 5 is substituted with A, C, D, F, G, H, K, M, N, Q, S, W, or Y; an amino acid Y at position 410 of SEQ ID NO: 5 remains unchanged or is substituted with A, C, D, G, N, S, T or V; an amino acid P at position 411 of SEQ ID NO: 5 remains unchanged or is substituted with A, C, G, I, L, M, N, S, T or V; and an amino acid A at position 486 of SEQ ID NO: 5 is substituted with C, D, E, F, G, H, I, K, L, M, N, P, Q, R, T, V, W or Y.

In one embodiment, the representative RNA polymerase variant is derived from Thermococcus litoralis DNA polymerase (Vent) having a wild-type amino acid sequence of SEQ ID NO: 6; and wherein: an amino acid L at position 411 of SEQ ID NO: 6 is substituted with A, C, D, F, G, H, K, M, N, Q, S, W, or Y; an amino acid Y at position 412 of SEQ ID NO: 6 remains unchanged or is substituted with A, C, D, G, N, S, T or V; and an amino acid P at position 413 of SEQ ID NO: 6 remains unchanged or is substituted with A, C, G, I, L, M, N, S, T or V.

In one embodiment, the representative RNA polymerase variant is derived from Thermococcus litoralis DNA polymerase (Vent) having a wild-type amino acid sequence of SEQ ID NO: 6; and wherein: an amino acid L at position 411 of SEQ ID NO: 6 is substituted with A, C, D, F, G, H, K, M, N, Q, S, W, or Y; an amino acid Y at position 412 of SEQ ID NO: 6 remains unchanged or is substituted with A, C, D, G, N, S, T or V; an amino acid P at position 413 of SEQ ID NO: 6 remains unchanged or is substituted with A, C, G, I, L, M, N, S, T or V; and an amino acid A at position 488 of SEQ ID NO: 6 is substituted with C, D, E, F, G, H, I, K, L, M, N, P, Q, R, T, V, W or Y.

In one embodiment, the representative RNA polymerase variant is derived from Methanosarcina acetivorans DNA polymerase (Mac) having a wild-type amino acid sequence of SEQ ID NO: 7; and wherein: an amino acid L at position 485 of SEQ ID NO: 7 is substituted with A, C, D, F, G, H, K, M, N, Q, S, W, or Y; an amino acid Y at position 486 of SEQ ID NO: 7 remains unchanged or is substituted with A, C, D, G, N, S, T or V; and an amino acid P at position 487 of SEQ ID NO: 7 remains unchanged or is substituted with A, C, G, I, L, M, N, S, T or V.

In one embodiment, the representative RNA polymerase variant is derived from Methanosarcina acetivorans DNA polymerase (Mac) having a wild-type amino acid sequence of SEQ ID NO: 7; and wherein: an amino acid L at position 485 of SEQ ID NO: 7 is substituted with A, C, D, F, G, H, K, M, N, Q, S, W, or Y; an amino acid Y at position 486 of SEQ ID NO: 7 remains unchanged or is substituted with A, C, D, G, N, S, T or V; an amino acid P at position 487 of SEQ ID NO: 7 remains unchanged or is substituted with A, C, G, I, L, M, N, S, T or V; and an amino acid A at position 565 of SEQ ID NO: 7 is substituted with C, D, E, F, G, H, I, K, L, M, N, P, Q, R, T, V, W or Y.

In one embodiment, the representative RNA polymerase variant is derived from Pyrobaculum islandicum DNA polymerase (Pis) having a wild-type amino acid sequence of SEQ ID NO: 8; and wherein: an amino acid M at position 426 of SEQ ID NO: 8 is substituted with A, C, D, F, G, H, K, N, Q, S, W, or Y; an amino acid Y at position 427 of SEQ ID NO: 8 remains unchanged or is substituted with A, C, D, G, N, S, T or V; and an amino acid P at position 428 of SEQ ID NO: 8 remains unchanged or is substituted with A, C, G, I, L, M, N, S, T or V.

In one embodiment, the representative RNA polymerase variant is derived from Pyrobaculum islandicum DNA polymerase (Pis) having a wild-type amino acid sequence of SEQ ID NO: 8; and wherein: an amino acid M at position 426 of SEQ ID NO: 8 is substituted with A, C, D, F, G, H, K, N, Q, S, W, or Y; an amino acid Y at position 427 of SEQ ID NO: 8 remains unchanged or is substituted with A, C, D, G, N, S, T or V; an amino acid P at position 428 of SEQ ID NO: 8 remains unchanged or is substituted with A, C, G, I, L, M, N, S, T or V; and an amino acid A at position 508 of SEQ ID NO: 8 is substituted with C, D, E, F, G, H, I, K, L, M, N, P, Q, R, T, V, W or Y.

In one embodiment, the representative RNA polymerase variant is derived from Sulfolobus solfataricus DNA polymerase (Sso) having a wild-type amino acid sequence of SEQ ID NO: 9; and wherein: an amino acid L at position 518 of SEQ ID NO: 9 is substituted with A, C, D, F, G, H, K, M, N, Q, S, W, or Y; an amino acid Y at position 519 of SEQ ID NO: 9 remains unchanged or is substituted with A, C, D, G, N, S, T or V; and an amino acid P at position 520 of SEQ ID NO: 9 remains unchanged or is substituted with A, C, G, I, L, M, N, S, T or V.

In one embodiment, the representative RNA polymerase variant is derived from Sulfolobus solfataricus DNA polymerase (Sso) having a wild-type amino acid sequence of SEQ ID NO: 9; and wherein: an amino acid L at position 518 of SEQ ID NO: 9 is substituted with A, C, D, F, G, H, K, M, N, Q, S, W, or Y; an amino acid Y at position 519 of SEQ ID NO: 9 remains unchanged or is substituted with A, C, D, G, N, S, T or V; an amino acid P at position 520 of SEQ ID NO: 9 remains unchanged or is substituted with A, C, G, I, L, M, N, S, T or V; and an amino acid A at position 601 of SEQ ID NO: 9 is substituted with C, D, E, F, G, H, I, K, L, M, N, P, Q, R, T, V, W or Y.

In one embodiment, the representative RNA polymerase variant exhibits an activity of synthesizing nucleic acids in a template-independent manner by adding at least one nucleotide selected from the group of naturally occurring nucleotide, nucleotide analogue, or a mixture thereof, to an extendible initiator.

In one embodiment, the extendible initiator comprises a single-stranded oligonucleotide initiator, a blunt-ended double-stranded oligonucleotide initiator, or a mixture thereof.

In one embodiment, the extendible initiator is a free form nucleic acid to be reacted in a liquid phase.

In one embodiment, the extendible initiator is immobilized on a solid support, wherein the solid support comprises a particle, bead, slide, array surface, membrane, flow cell, well, microwell, nano-well, chamber, microfluidic chamber, channel, or microfluidic channel.

In one embodiment, the at least one nucleotide is linked with a detectable label.

In one embodiment, the at least one nucleotide comprises a ribose.

In one embodiment, the representative RNA polymerase variant exhibits the activity at reaction temperatures ranging from 10° C. to 100° C.

In one aspect, the present disclosure further provides a kit for performing de novo enzymatic nucleic acid synthesis, comprising an RNA polymerase variant derived from a wild-type B-family DNA polymerase having an amino acid sequence selected from the group consisting of SEQ ID NO: 2, 3, 4, 5, 6, 7, 8, 9, 10, 11, 12, 13, 14, 15, 16, and 17, wherein the RNA polymerase variant exhibits activity of synthesizing nucleic acids in a template-independent manner by adding at least one nucleotide selected from the group of naturally occurring nucleotide, nucleotide analogue, or a mixture thereof, to an extendible initiator, thereby synthesizing a desired nucleic acid sequence.

In another aspect, the present disclosure further provides method for template-independent synthesis of an RNA oligonucleotide, comprising:
(a) providing an initiator oligonucleotide,
(b) providing an RNA polymerase variant;
(c) combining the initiator oligonucleotide, the RNA polymerase variant and one or more nucleotides under conditions sufficient for the addition of at least one nucleotide to the 3′ end of the initiator oligonucleotide;
   wherein the selected RNA polymerase variant comprising:
   a motif A, and a motif B corresponding to positions 706 to 730, and 843 to 855, respectively, of a consensus sequence (SEQ ID NO:1); and at least one amino acid substitution at a position in the motif A, the motif B, or the combination thereof; wherein the RNA polymerase variant has a reduced or eliminated the intrinsic 3′ to 5′ exonuclease activity.

Accordingly, the present invention relates to the specific RNA polymerase variants that exhibit an improved performance on incorporating a variety of nucleotides for nucleic acid synthesis at various reaction temperatures in the absence of nucleic acid template. More particularly, the de novo RNA synthesis method can be efficiently performed by said RNA polymerase variants with a broad-spectrum of nucleotides and nucleotide analogues.

### BRIEF DESCRIPTION OF THE DRAWINGS

The present disclosure will become more readily appreciated with reference to the following description in conjunction with the accompanying drawings.

FIG. 1 shows an amino acid sequence alignment of the wild-type B-family DNA polymerases (PolB) related to the present invention and their consensus sequence.

FIGS. 2A and 2B show the results of the RNA synthesis reactions described in Example 3.1.

FIGS. 3A and 3B show the results of the RNA synthesis reactions described in Example 3.2.

FIGS. 4A and 4B show the results of the RNA synthesis reactions described in Example 3.3.

FIGS. 5A and 5B show the results of the RNA synthesis reactions described in Example 3.4.

FIGS. 6A and 6B show the results of the RNA synthesis reactions described in Example 3.5.

### DETAILED DESCRIPTION

### Definition

All terms including descriptive or technical terms which are used herein should be construed as having meanings that are understandable to one of ordinary skill in the art. However, the terms may have different meanings according to an intention of the user, case precedents, or the appearance of new technologies. Also, some terms may be arbitrarily selected by the applicant, and in this case, the meaning of the selected terms will be described in detail in the descriptions of the present disclosure. Thus, the terms used herein are defined based on the meaning of the terms together with the descriptions throughout the specification. In addition, the titles and subtitles may be attached to the description for readability, but these titles do not affect the scope of the present invention.

As used herein, the term “a,” “an,” or “the” includes plural referents unless expressly and unequivocally limited to one referent. The term “or” is used interchangeably with the term “and/or” unless the context clearly indicates otherwise.

Also, when a part or a method “includes” or “comprises” a component or a step, respectively, unless there is a particular description contrary thereto, the part or the method can further include other components or other steps, not excluding the others.

As used herein, an “amino acid” refers to any monomer unit that can be incorporated into a peptide, polypeptide, or protein. As used herein, the term “amino acid” includes the following twenty natural or genetically encoded alpha-amino acids: alanine (Ala or A), arginine (Arg or R), asparagine (Asn or N), aspartic acid or aspartate (Asp or D), cysteine (CyS or C), glutamine (Gln or Q), glutamic acid or glutamate (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). In cases where “X” residues are undefined, these should be defined as “any amino acid”.

The term “functionally equivalent” or “equivalent” is used to describe a specific B-family DNA polymerase (PolB) variant and any of the derivative RNA polymerase variant provided herein having the substitution or mutation that is considered to occur at the amino acid position in the other PolB, PolB variant according to the sequence alignment, or a reference sequence, which has the same functional or structural role in the enzyme. The equivalent positions may be defined according to homologues, conserved motifs, user-defined, or derived, consensus sequence.

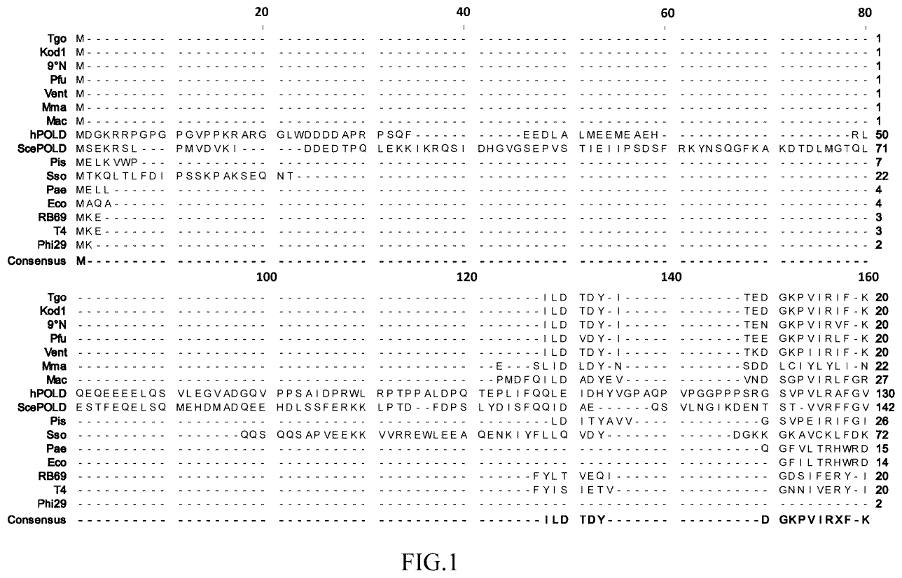

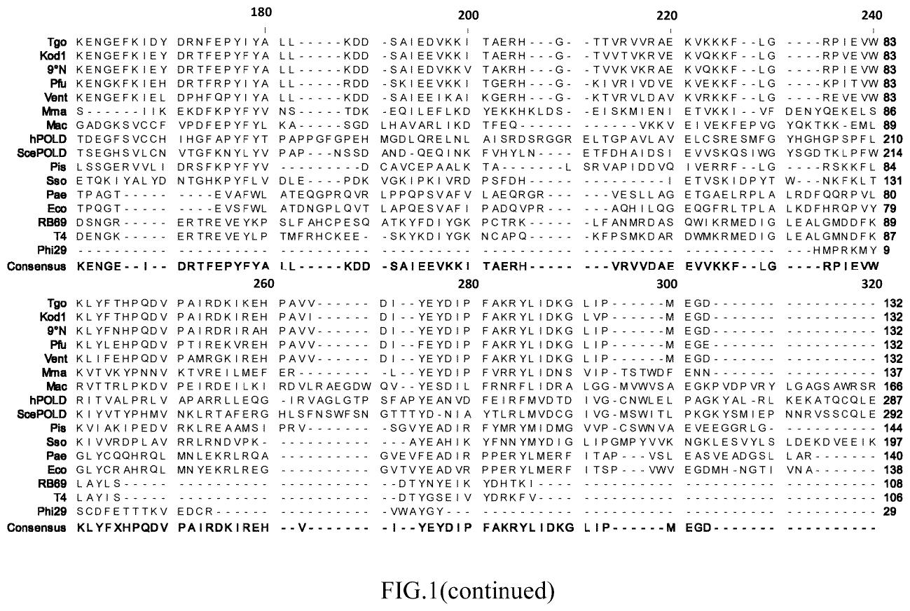

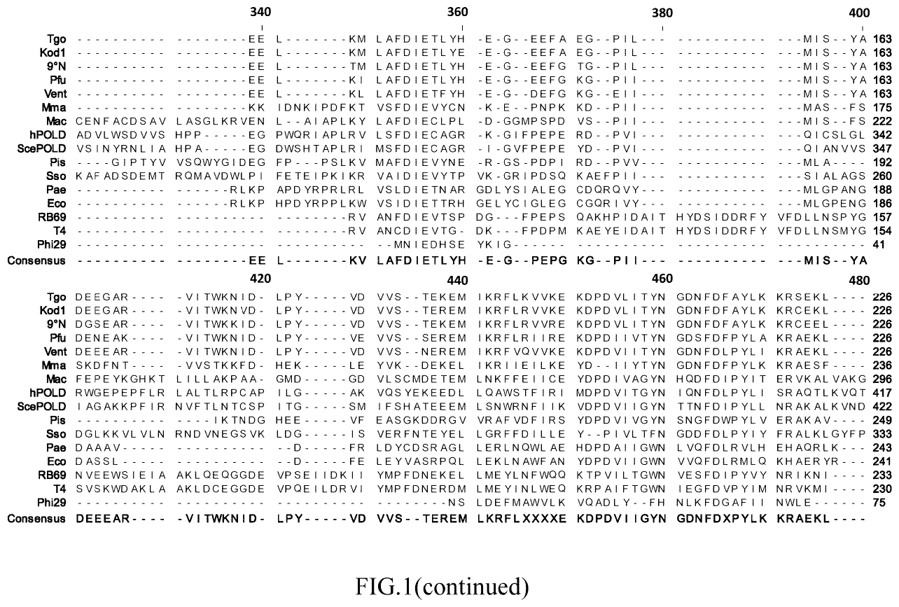

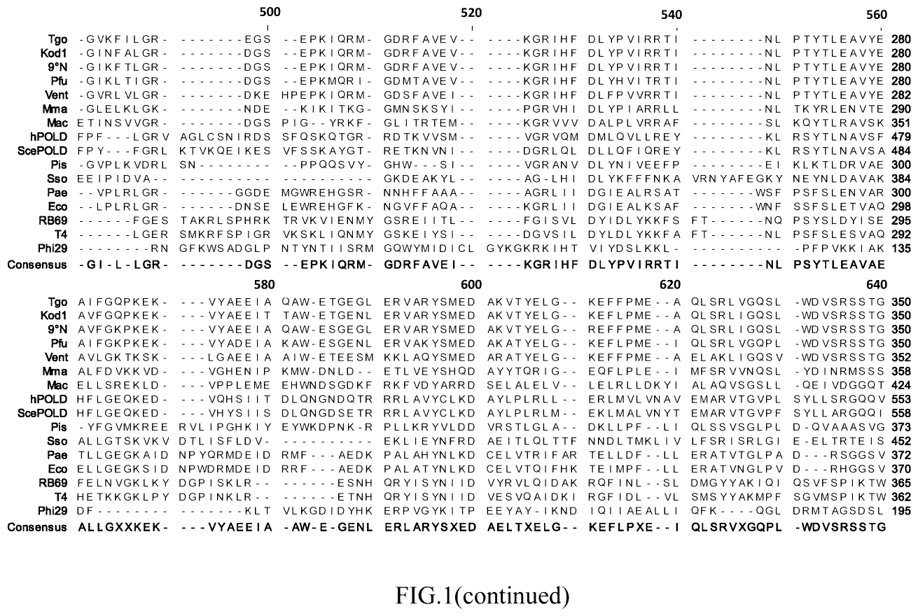

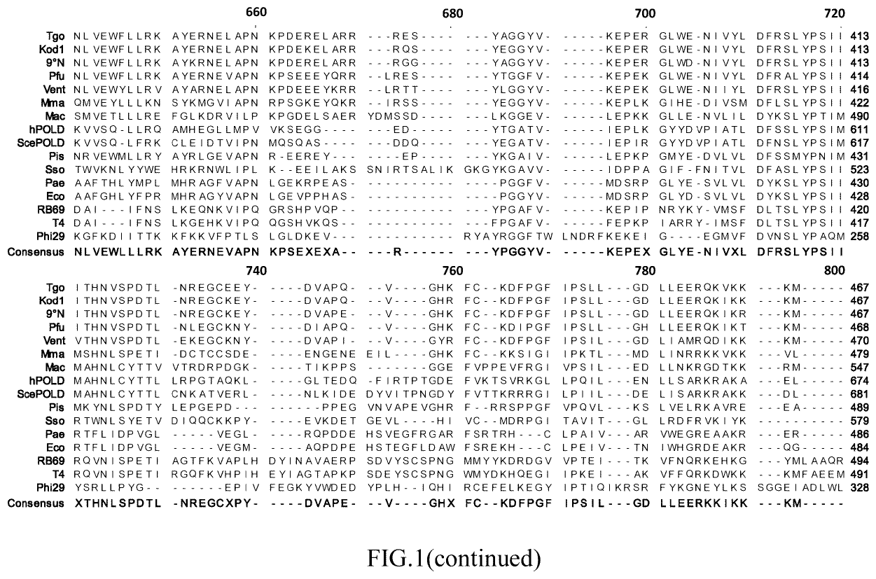

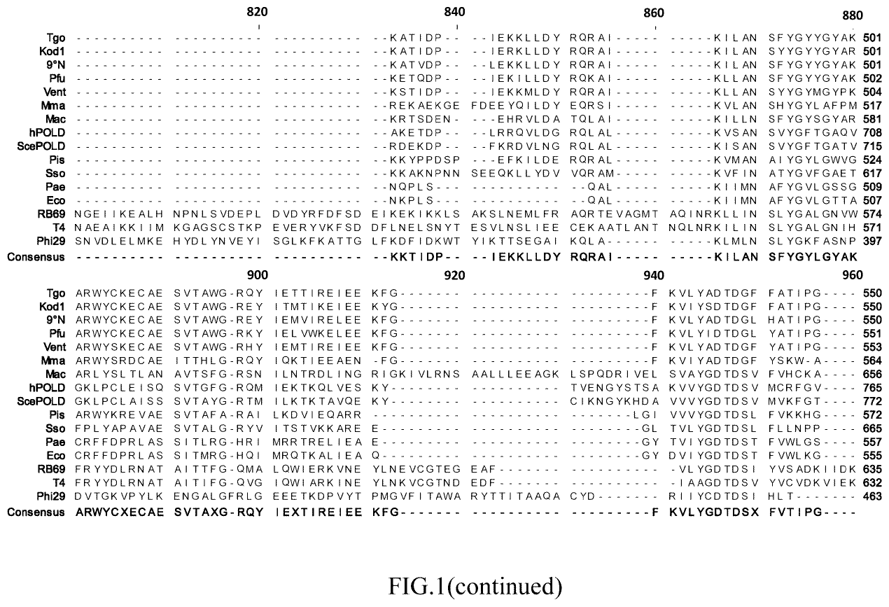

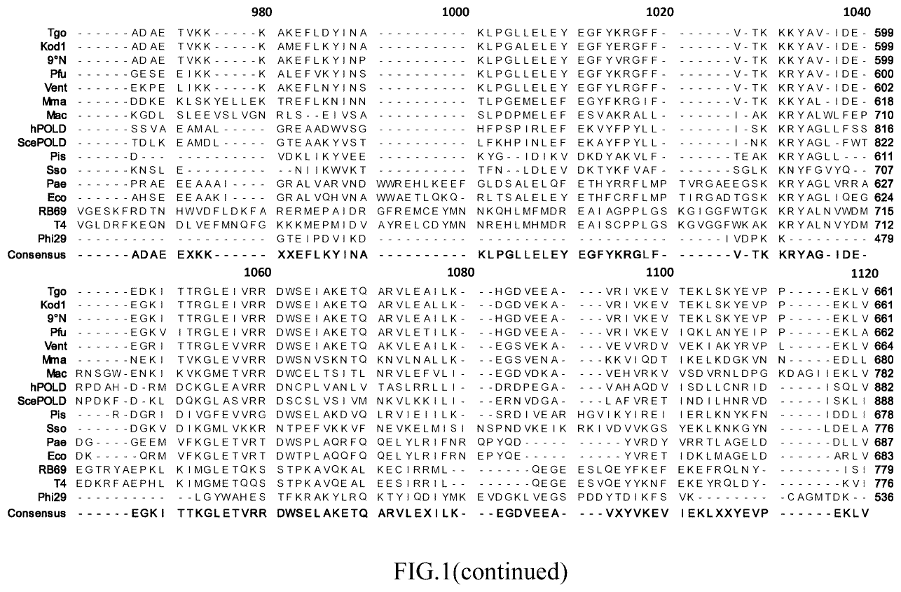

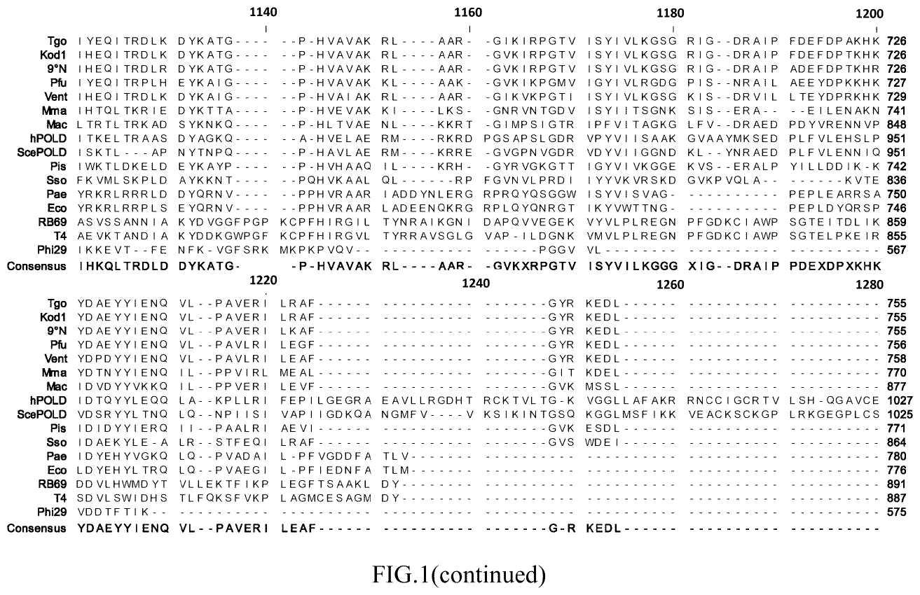

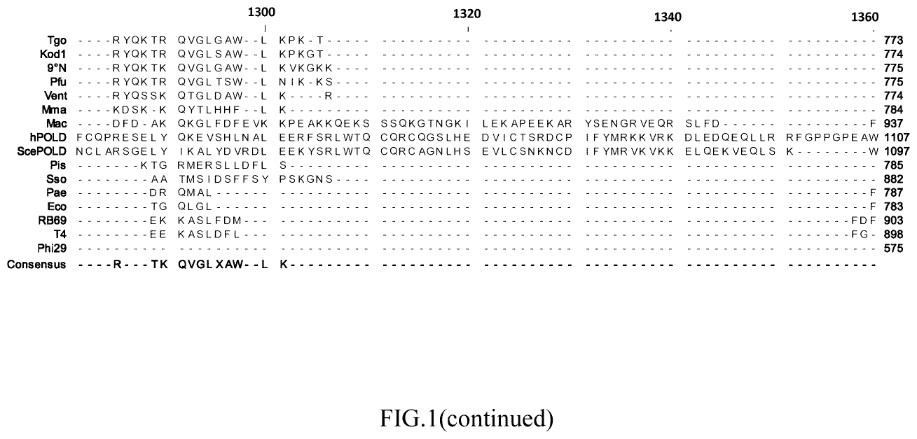

Generally, the homologous PolBs have similar or identical amino acid sequences and functional structure, and thereby the equivalent amino acid substitution mutations among different PolBs generally occur at homologous amino acid positions. The term “functionally equivalent” or “equivalent” used herein also encompass mutations that are “homologous” or “positionally equivalent” to a given mutation in view of protein sequence or structural alignment, regardless of the actual function of the mutated amino acid. Practically, the “functionally equivalent”, “homologous” and/or “positionally equivalent” amino acid residues of different polymerases can be identified according to the protein sequence or structural alignment. Accordingly, a cross-species alignment was made on multiple wild-type PolBs, as illustrated in FIG. 1, and the consensus sequence (SEQ ID NO: 1) is used as a positional reference sequence.

For example, the substitution of amino acid aspartic acid (D) with alanine (A) at position 141 of the wild-type Thermococcus kodakarensis (Kod1) (D141A) amino acid sequence would be functionally equivalent to the amino acid substitution mutation D114A at the conserved residue of wild-type Escherichia coli phage RB69 DNA polymerase (RB69) amino acid sequence. When the positional reference sequence is used to describe these equivalent amino acid substitutions, the functionally equivalent positions of both amino acid residue 141 of Kod1 and amino acid residue 114 of RB69 corresponds to position 354 of the consensus sequence (SEQ ID NO: 1).

The term “conserved” means the segment of polymerase having the same amino acid residue in the homologous or equivalent position of different PolBs from various sources. The term “semi-conserved” used herein refers to the segment of polymerase that has a similar property of amino acid residue or an identical amino acid residue in the homologous position of different PolBs from various sources.

The terms “nucleic acid”, “nucleic acid sequence”, “oligonucleotide”, “polynucleotide”, and “nucleic acid fragment” as used herein refer to a deoxyribonucleotide or ribonucleotide sequence in a single-stranded or a double-stranded form of which the sources and length are not limited herein; and generally, includes naturally occurring nucleotides or artificial chemical mimics. The term “nucleic acid” as used herein is interchangeable with the terms including natural or unnatural “oligonucleotide”, “polynucleotide”, “DNA”, “RNA”, “gene”, “complementary DNA” (cDNA), and “messenger RNA” (mRNA) in use.

The “nucleic acid”, “oligonucleotide”, or “polynucleotide” used herein refers to a polymer that can be corresponded to a ribose nucleic acid (RNA) or deoxyribose nucleic acid (DNA) polymer, or an analogue thereof. This includes polymers of nucleotides such as RNA and DNA, as well as synthetic forms, modified (e.g., chemically or biochemically modified) forms thereof, and mixed polymers (e.g., including both RNA and DNA subunits). Exemplary modifications include methylation, substitution of one or more of the naturally occurring nucleotides with an analog, internucleotide modifications such as uncharged linkages (e.g., methyl phosphonates, phosphotriesters, phosphoamidates, carbamates, and the like), pendentmoieties (e.g., polypeptides), intercalators (e.g., acridine, psoralen, and the like), chelators, alkylators, and modified linkages (e.g., alpha anomeric nucleic acids and the like). Also included are synthetic molecules that mimic polynucleotides in their ability to bind to a designated sequence via hydrogen bonding and other chemical interactions. Typically, the nucleotide monomers are linked via phosphodiester bonds, although synthetic forms of nucleic acids can comprise other linkages (e.g., peptide nucleic acids as described in Nielsen et al. Science 254:1497-1500, 1991). A nucleic acid can be or can include, e.g., a chromosome or chromosomal segment, a vector (e.g., an expression vector), an expression cassette, a naked DNA or RNA polymer, the product of a polymerase chain reaction (PCR), an oligonucleotide, a probe, and a primer. A nucleic acid can be, e.g., single-stranded, double-stranded, or triple-stranded and is not limited to any particular length. Unless otherwise indicated, a particular nucleic acid sequence optionally comprises or encodes complementary sequences, in addition to any sequence explicitly indicated.

The nucleic acid as used herein also includes nucleic acid analogue. The term nucleic acid analogue is known to describe compounds or artificial nucleic acids which are functionally or structurally equivalent to naturally existing RNA and DNA. A nucleic acid analogue may have one or more parts of a nucleotide (the phosphate backbone, pentose sugar, and nucleobase) being modified. These modifications on the nucleotide change the structure and geometry of the nucleic acid and its interactions with nucleic acid polymerases. The nucleic acid analogue also encompasses the emerging category of artificial nucleic acids, such as XNA, which is designed to have new-to-nature forms of sugar backbone.

Examples of nucleic acid analogues include but are not limited to: the universal bases, such as inosine, 3-nitropyrrole, and 5-nitroindole, which can form a base-pair with all four canonical bases; the phosphate-sugar backbone analogues, such as peptide-nucleic acids (PNA), which affect the backbone properties of the nucleic acid; chemical linker or fluorophore-attached analogues, such as amine-reactive aminoallyl nucleotide, thiol-containing nucleotides, biotin-linked nucleotides, rhodamine-linked nucleotides, and cyanine-linked nucleotides; the fluorescent base analogues, such as 2-aminopurine (2-AP), 3-methylisoxanthopterin (3-MI), 3-methylisoxanthopterin (6-MI), 4-amino-6-methylisoxanthopterin (6-MAP), and 4-dimethylaminopyridine (DMAP); the nucleic acid probes for various genetic applications, such as the oligonucleotide-conjugated with a fluorescent reporter dye (ALEXA, FAM, TET, TAMRA, CY3, CY5, VIC, JOE, HEX, NED, PET, ROX, Texas Red and others) and/or a fluorescent quenchers (BHQs); the molecular beacons (MBs), which are single-stranded nucleic acid probes containing a stem-loop structure and a dual fluorophore-and-quencher label; and the nucleic acid aptamers.

Generally, as used herein, a “template” is a polynucleotide, or a polynucleotide mimic, that contains the desired or unknown target nucleotide sequence. In some instances, the terms “target sequence”, “template polynucleotide”, “target nucleic acid”, “target polynucleotide”, “nucleic acid template”, “template sequence, and variations thereof, are used interchangeably. Specifically, the term “template” refers to a strand of nucleic acid from which a complimentary copy is synthesized from nucleotides or nucleotide analogues through the replication by a template-dependent or template-directed nucleic acid polymerase. Within a nucleic acid duplex, the template strand is, by the convention definition, depicted and described as the “bottom” strand. Similarly, the non-template strand is often depicted and described as the “top” strand. The “template” strand may also be referred to as the “sense”, or “plus”, strand and the non-template strand as the “antisense”, or “minus”, strand.

The term “initiator” refers to a mononucleoside, a mononucleotide, and oligonucleotide, a polynucleotide, or modified analogues thereof, from which a nucleic acid is to be synthesized by nucleic acid polymerase de novo. The term “initiator” may also refer to a xeno nucleic acids (XNA) or a peptide nucleic acid (PNA) having a 3′-hydroxyl group.

The terms “nucleotide incorporation”, “analogue incorporation”, “incorporating nucleotide” and “incorporating analogue” are known to those skilled in the art and used to describe a process or reaction for nucleic acid synthesis. Thus, as used herein, the term “incorporation” is known to flexibly refer to add one, or more nucleotides, or any specified nucleic acid precursors to the 3′-hydroxyl terminus of a nucleic acid initiator or a primer.

The term “nucleotide analogue” is known to those skilled in the art to describe the chemically modified nucleotides or artificial nucleotides, which are structural mimics of canonical nucleotides. These nucleotide analogues can serve as substrates for nucleic acid polymerases to synthesize nucleic acid. A nucleotide analogue may have one or more altered components of a nucleotide (e.g., the phosphate backbone, pentose sugar, and nucleobase), which changes the structure and configuration of a nucleotide and affects its interactions with other nucleobases and the nucleic acid polymerases. For example, a nucleotide analogue having altered nucleobase may confer alternative base-pairing and base-stacking properties in the DNA or RNA. Furthermore, by way of example, the modification at the base may generate various nucleosides such as inosine, methyl-5-deoxycytidine, deoxyuridine, dimethylamino-5-deoxyuridine, diamino-2,6-purine or bromo-5-deoxyuridine, and any other analogues which permits hybridization. In other exemplary aspects, modifications may take place at the level of sugar moiety (for example, replacement of a deoxyribose by an analogue), and/or at the level of the phosphate group (for example, boronate, alkylphosphonate, or phosphorothioate derivatives). A nucleotide analogue monomer may have a phosphate group selected from a monophosphate, a diphosphate, a triphosphate, a tetraphosphate, a pentaphosphate, and a hexaphosphate.

Other examples of nucleotide analogues also include nucleotides having a removable blocking moiety. Examples of the removable blocking moiety include, but are not limited to, a 3′-O-blocking moiety, a base blocking moiety, and a combination thereof. Examples of the 3′-O-blocking moiety include, but are not limited to, O-azido (O—N₃), O-azidomethyl, O-amino, O-allyl, O-phenoxyacetyl, O-methoxyacetyl, O-acetyl, O-(p-toluene)sulfonate, O-phosphate, O-nitrate, O-[4-methoxy]-tetrahydrothiopyranyl, O-tetrahydrothiopyranyl, O-[5-methyl]-tetrahydrofuranyl, O-[2-methyl, 4-methoxy]-tetrahydropyranyl, O-[5-methyl]-tetrahydropyranyl, and O-tetrahydrothiofuranyl, O-2-nitrobenzyl, O-methyl, and O-acyl groups. Examples of the base blocking moiety may be a reversible dye-terminator. Examples of the reversible dye-terminator include, but are not limited to, a reversible dye-terminator of Illumina MiSeq, a reversible dye-terminator of Illumina HiSeq, a reversible dye-terminator of Illumina Genome Analyzer IIX, a reversible dye-terminator of Helicos Biosciences Heliscope, and a reversible dye-terminator of LaserGen's Lightning Terminators.

As used herein, “B-family DNA polymerases (PolBs)” refers to the most common template-dependent DNA polymerases or replicases in all domains of life and many DNA viruses. Like most nucleic acid polymerases, natural PolBs require a duplex primer-template DNA with a free 3′-hydroxyl (3′-OH) group at the primer terminus, all four nucleoside triphosphates (dATP, dTTP, dCTP, and dGTP), and catalytic divalent cations (Mg²⁺ or Mn²⁺, etc.) for catalyzing the nucleotidyl transferase reaction of adding nucleotides to the 3′-OH terminus of a primer. The PolB enzymes, such as bacterial Pol II and archaeal B-family DNA polymerases, are replicative and repair polymerases that inherently contain a catalytic polymerase domain and a 3′ to 5′ exonucleolytic, or proofreading, domain for removing the mis-incorporated nucleotide from the growing primer strand during nucleic acid replication. The term “3′ to 5′ exonucleolytic domain” (Exo domain) refers to a region of the amino acid sequence of a polymerase, which exerts the nucleic acid degradation activity from the 3′-terminus of the primer or the polynucleotide chain. Coordinately, the term “catalytic polymerase domain” (Pol domain) refers to a region of the amino acid sequence of a polymerase, which exerts the catalytic DNA/RNA polymerase activity for adding nucleotides to the 3′-terminus of a primer or a polynucleotide chain.

All known structures of PolB catalytic polymerase domain resemble the shape of human right hand, where the key functional regions are characterized as fingers, palm, and thumb subdomains. The most conserved region is the palm subdomain, which contains the essential residues for catalysis. The protein sequence-alignment among various B-family DNA polymerases from different kingdoms of life and DNA viruses reveals that the PolB polymerases generally harbor six semi-conserved or conserved motifs (I-VI) for their essential exonuclease and polymerase functions. The first three sequence-motifs (Exo I, Exo II, Exo III) are in the Exo domain, while the other three motifs (designated as Motif A, B, and C, respectively) reside in the Pol domain (Hopfner et al, Proc. Natl. Acad. Sci. USA 96, 3600-3605, 1999). In some embodiments of the present invention, without being limited by any theory, it is discovered that through modifying some novel positions/regions in said motifs of B-family DNA polymerases, the polymerases can thereby effectively catalyze de novo RNA synthesis as a template-independent RNA polymerases. Based on the acquired activities of de novo RNA synthesis, these modified B-family DNA polymerases are referred to as template-independent RNA polymerase variants. In other words, the RNA polymerase variant is capable of incorporating ribonucleotides, such as rATP, rUTP, rCTP and rGTP, at the 3′-terminus of an initiator oligonucleotide under impeding reaction conditions.

As used herein, the term “mutant” in the context of the present invention, means a polypeptide, typically recombinant, that comprises one or more amino acid substitutions relative to a corresponding, functional DNA polymerase.

As used herein, in the context of the present invention, “corresponding to another sequence” (e.g., regions, fragments, nucleotide or amino acid positions, or the like) is based on the convention of numbering according to nucleotide or amino acid position number and then aligning the sequences in a manner that maximizes the percentage of sequence identity. An amino acid “corresponding to position X of specific sequence” refers to an amino acid in a polypeptide of interest that aligns with the equivalent amino acid of a specified sequence. Generally, as described herein, the amino acid corresponding to a position of a polymerase can be determined using an alignment algorithm such as BLAST and other currently available tools for conducting amino acid sequence alignment. Because not all positions within a given “corresponding region” need to be identical, non-matching positions within a corresponding region may be regarded or define as “corresponding positions”. Accordingly, as used herein, referral to an “amino acid position corresponding to amino acid position X of a specified DNA polymerase” refers to equivalent positions, based on alignment, in other DNA polymerases and structural homologues and families.

As used herein, the term “consensus sequence of SEQ ID NO: 1” used herein refers to a reference sequence comprising the conserved or semi-conserved amino acids of cross-species B-family DNA polymerase. The consensus sequence of SEQ ID NO: 1 is a virtual sequence and is generated by aligned the following 16 wild-type B-family DNA polymerases to obtain the conserved amino acids: Thermococcus gorgonarius DNA polymerase (Tgo), Thermococcus kodakarensis DNA polymerase (Kod1), Thermococcus sp. (strain 9° N-7) DNA polymerase (9° N), Pyrococcus furiosus DNA polymerase (Pfu), Thermococcus litoralis DNA polymerase (Vent), Methanococcus maripaludis DNA polymerase (Mma), Methanosarcina acetivorans DNA polymerase (Mac), human DNA polymerase delta catalytic p125 subunit (hPOLD), Saccharomyces cerevisiae DNA polymerase delta catalytic subunit (ScePOLD), Pyrobaculum islandicum DNA polymerase (Pis), Sulfolobus solfataricus DNA polymerase (Sso), Pseudomonas aeruginosa DNA polymerase II (Pae), Escherichia. coli DNA polymerase II (Eco), Escherichia coli phage RB69 DNA polymerase (RB69), Escherichia coli phage T4 DNA polymerase (T4), or Bacillus phage Phi29 DNA polymerase (Phi29). These PolB sequences are aligned for obtaining the alignment sequence as a reference of functionally equivalent positions.

The positions of motifs Exo I, Exo II, Exo III, A, B, and C are defined by the inventor using the consensus sequence of SEQ ID NO: 1 of the present invention; therefore, it shall be noted that the positions of these motifs defined in the present invention are not totally the same as those described in the literature or prior art.

### OBJECTIVES

The inventor has discovered PolB variants that have an improved function and activity for utilizing canonical nucleotides, nucleotide analogues, and initiators for synthesizing polynucleotides in a template-independent manner. These PolB variants can efficiently add said canonical nucleotides or nucleotide analogues to said initiator in the absence of a replicative template to synthesize a polynucleotide with a random or defined sequence.

More specifically, the inventor has discovered PolB variants can efficiently catalyze the additions of natural ribonucleotides (rNTP), such as rATP, rUTP, rCTP and rGTP, to the 3′-OH ends of a single-stranded nucleic acid initiator or a blunt-end duplex nucleic acid initiator, in the absence of replicative template, to generate polynucleotides with desired nucleic acid sequences. Furthermore, the PolB variants provided herein generally have a broader substrate specificity, which means the PolB variants can utilize not only naturally occurring nucleotides, but also varieties of modified nucleotides and nucleic acid analogues for the de novo nucleic acid synthesis. Thus, the modified nucleotide can be further designed for being incorporated to the initiator to generate certain functional polynucleotides. Therefore, these PolB variants broaden the scope and utility of template-independent enzymatic nucleic acid synthesis applications for synthesizing polynucleotides with desired sequences and features.

### Protein Sequence Alignment of B-Family DNA Polymerases

FIG. 1 shows the amino acid sequence alignment of 16 different wild-type B-family DNA polymerases (PolBs) utilized by the inventor, and the outcome of consensus sequence alignment is listed in the bottom (SEQ ID NO:1). The 16 wild-type PolBs used for alignment are Thermococcus gorgonarius DNA polymerase (Tgo, SEQ ID NO:2), Thermococcus kodakarensis DNA polymerase (Kod1, SEQ ID NO:3), Thermococcus sp. (strain 9° N-7) DNA polymerase (9° N, SEQ ID NO:4), Pyrococcus furiosus DNA polymerase (Pfu, SEQ ID NO:5), Thermococcus litoralis DNA polymerase (Vent, SEQ ID NO:6), Methanosarcina acetivorans DNA polymerase (Mac, SEQ ID NO:7), Pyrobaculum islandicum DNA polymerase, (Pis, SEQ ID NO:8), Sulfolobus solfataricus DNA polymerase (Sso, SEQ ID NO:9), Methanococcus maripaludis DNA polymerase (Mma, SEQ ID NO:10), human DNA polymerase delta catalytic p125 subunit (hPOLD, SEQ ID NO:11), Saccharomyces cerevisiae DNA polymerase delta catalytic subunit (ScePOLD, SEQ ID NO:12), Pseudomonas aeruginosa DNA polymerase II (Pae, SEQ ID NO:13), Escherichia. coli DNA polymerase II (Eco, SEQ ID NO:14), Escherichia coli phage RB69 DNA polymerase (RB69, SEQ ID NO:15), Escherichia coli phage T4 DNA polymerase (T4, SEQ ID NO:16), and Bacillus phage Phi29 DNA polymerase (Phi29, SEQ ID NO:17).

As shown in FIG. 1, various sequence regions among these exemplary wild-type PolBs are highly conserved while other regions are more variable. Those of skill in the art will immediately recognize and understand that mutations in addition to those specifically identified and discussed herein may be also made in the variable regions of wild-type PolBs without altering, or without substantially altering, the polymerase activity of the mutated enzyme. Likewise, conservative mutations at conserved residues/positions of any of PolBs may be made without altering, or substantially altering, the polymerase activity of the mutated enzyme. Enzyme engineering based on comparative structure analysis with other functionally related enzymes or homologs is a useful technique in the molecular biology field that allows the inventor to reasonably predict the effect of a given mutation on the catalytic activity of the enzyme. Based on the present disclosure, using the sequence, structural data, and known physical properties of amino acids, those of skill in the art can mutate enzymes, such as the DNA polymerases encompassed by the present invention, without altering, or without substantially altering, the essential, intrinsic characteristics of the enzymes.

Besides, the motifs Exo I, Exo II, Exo III, A, B, and C corresponding to the positions 349 to 364, 450 to 476, 590 to 608, 706 to 730, 843 to 855, and 940 to 956 respectively, of the consensus sequence of SEQ ID NO:1 are focused in the present disclosure. More specifically, the polymerase variant in the present invention is based on substitution mutations at one or more residues correspondingly residing in said motifs.

### RNA Polymerase Variant

In view of the above, provided herein are altered polymerase, which is described based on the amino acid sequence of the consensus sequence of SEQ ID NO: 1. An altered polymerase includes substitution mutations at one or more residues when compared to the consensus sequence of SEQ ID NO: 1. A substitution mutation can be at the same, a homologous, or a functionally equivalent position as compared to the consensus sequence of SEQ ID NO: 1. The skilled person can readily appreciate that an altered polymerase described herein is not naturally occurring. Therefore, an altered polymerase described herein is based on the consensus sequence of SEQ ID NO: 1 and further includes substitution mutations at one or more residues of the corresponding wild-type polymerase (individual parent polymerase). In one embodiment, at least one substitution mutation is at a position functionally equivalent to an amino acid of the consensus sequence of SEQ ID NO: 1. “Functionally equivalent” means that the altered polymerase has the amino acid substitution at the amino acid position according to the consensus sequence of SEQ ID NO: 1 that has the same functional or structural role in both the consensus sequence and the altered polymerase.

In general, functionally equivalent substitution mutations in two or more different polymerases occur at homologous amino acid positions in the amino acid sequences of the polymerases. Hence, “functionally equivalent” also encompasses mutations that are “positionally equivalent” or “homologous” to a given mutation, regardless of whether or not the particular function of the mutated amino acid is known. It is possible to identify the regions of functionally equivalent and positionally equivalent amino acid residues in the amino acid sequences of two or more different polymerases on the basis of sequence alignment and/or molecular modelling. For instance, the amino acid sequence alignment of exemplary 16 wild-type B-family DNA polymerases from different domains of life are used to identify positionally equivalent and/or functionally equivalent residues. The result of the protein sequence alignment among these PolBs is set forth in FIG. 1. Thus, for the exemplary residue 141 of the Tgo, Kod1, 9° N, Pfu, and Vent polymerases, residue 171 of the Pis, residue 231 of the Sso, and residue 198 of the Mac polymerase are functionally equivalent and positionally equivalent. Likewise, for the exemplary residue 143 of the Tgo, Kod1, 9° N, Pfu, and Vent polymerases, residue 173 of the Pis, residue 233 of the Sso, and residue 200 of the Mac polymerase are functionally equivalent and positionally equivalent. The skilled person can easily identify functionally equivalent residues in different polymerases.

In accordance with some embodiments, the provided RNA polymerase variant comprising: a motif A, and a motif B corresponding to positions 706 to 730, and 843 to 855, respectively, of a consensus sequence (SEQ ID NO:1); and at least one amino acid substitution (one or more amino acid substitutions, or a combination of amino acid substitutions) at a position in the motif A, the motif B, or the combination thereof; wherein the RNA polymerase variant has reduced or deficient in the 3′ to 5′ exonuclease activity. Said the 3′ to 5′ exonuclease activity deficiency can be reached by any means. For example, practically, the 3′ to 5′ exonuclease activity can be reduced, attenuated, removed or inactivated by modifying the 3′ to 5′ exonucleolytic domain of the polymerase to generate a polymerase that has reduced or is deficient in the 3′ to 5′ exonuclease activity. Preferably, the means of amino acid substitution is adapted to modify the 3′ to 5′ exonucleolytic domain. In other words, the provided RNA polymerase variant may comprise at least one amino acid substitution in Exo I, Exo II, Exo III or the combination. For example, the PolB variants may have functionally equivalent or positionally equivalent substitutions of the native D with A at position 354 (D354A) and the native E with A at position 356 (E356A) in the motif Exo I of SEQ ID NO:1, thereby causing a 3′-5′ exonuclease deficiency.

In accordance with some embodiments, the RNA polymerase variant is modified from a wild-type B-family DNA polymerase having an amino acid sequence selected from the group consisting of SEQ ID NO: 2, 3, 4, 5, 6, 7, 8, 9, 10, 11, 12, 13, 14, 15, 16 and 17., which are respectively derived from the wild-type B-family DNA polymerase of Thermococcus gorgonarius DNA polymerase (Tgo), Thermococcus kodakarensis DNA polymerase (Kod1), Thermococcus sp. (strain 9° N-7) DNA polymerase (9° N), Pyrococcus furiosus DNA polymerase (Pfu), Thermococcus litoralis DNA polymerase (Vent), Methanosarcina acetivorans DNA polymerase (Mac), Pyrobaculum islandicum DNA polymerase (Pis), Sulfolobus solfataricus DNA polymerase (Sso), Methanococcus maripaludis DNA polymerase (Mma), human DNA polymerase delta catalytic p125 subunit (hPOLD), Saccharomyces cerevisiae DNA polymerase delta catalytic subunit (SecPOLD), Pseudomonas aeruginosa DNA polymerase II (Pae), Escherichia. coli DNA polymerase II (Eco), Escherichia phage RB69 DNA polymerase (RB69), Escherichia phage T4 DNA polymerase (T4), and Bacillus phage Phi29 DNA polymerase (Phi29).

In accordance with certain embodiments, the representative RNA polymerase variant comprises a motif Exo I corresponding to positions 349 to 364 of the consensus sequence (SEQ ID NO:1), and the RNA polymerase variant has at least one amino acid substitution at a position in the motif Exo I. Preferably, an amino acid L or M corresponding to position 715 of SEQ ID NO: 1 is substituted with A, C, D, F, G, H, K, N, Q, S, W, or Y; an amino acid Y corresponding to position 716 of SEQ ID NO: 1 remains unchanged or is substituted with A, C, D, G, N, S, T or V; and an amino acid P corresponding to position 717 of SEQ ID NO: 1 remains unchanged or is substituted with A, C, G, I, L, M, N, S, T or V.

In accordance with some embodiments, the representative RNA polymerase variant is derived from Thermococcus gorgonarius DNA polymerase (Tgo) having a wild-type amino acid sequence of SEQ ID NO: 2; and wherein: an amino acid L at position 408 of SEQ ID NO: 2 is substituted with A, C, D, F, G, H, K, M, N, Q, S, W, or Y, preferably Y; an amino acid Y at position 409 of SEQ ID NO: 2 remains unchanged or is substituted with A, C, D, G, N, S, T or V, preferably A; and an amino acid P at position 410 of SEQ ID NO: 2 remains unchanged or is substituted with A, C, G, I, L, M, N, S, T or V, preferably G.

In accordance with some embodiments, the representative RNA polymerase variant is derived from Thermococcus gorgonarius DNA polymerase (Tgo) having a wild-type amino acid sequence of SEQ ID NO: 2; and wherein: an amino acid L at position 408 of SEQ ID NO: 2 is substituted with A, C, D, F, G, H, K, M, N, Q, S, W, or Y, preferably Y; an amino acid Y at position 409 of SEQ ID NO: 2 remains unchanged or is substituted with A, C, D, G, N, S, T or V, preferably A; an amino acid P at position 410 of SEQ ID NO: 2 remains unchanged or is substituted with A, C, G, I, L, M, N, S, T or V, preferably G; and an amino acid A at position 485 of SEQ ID NO: 2 is substituted with C, D, E, F, G, H, I, K, L, M, N, P, Q, R, T, V, W or Y, preferably E or L.

In accordance with some embodiments, the representative RNA polymerase variant is derived from Thermococcus kodakarensis DNA polymerase (Kod1) having a wild-type amino acid sequence of SEQ ID NO: 3; and wherein: an amino acid L at position 408 of SEQ ID NO: 3 is substituted with A, C, D, F, G, H, K, M, N, Q, S, W, or Y, preferably Y; an amino acid Y at position 409 of SEQ ID NO: 3 remains unchanged or is substituted with A, C, D, G, N, S, T or V, preferably A; and an amino acid P at position 410 of SEQ ID NO: 3 remains unchanged or is substituted with A, C, G, I, L, M, N, S, T or V, preferably G.

In accordance with some embodiments, the representative RNA polymerase variant is derived from Thermococcus kodakarensis DNA polymerase (Kod1) having a wild-type amino acid sequence of SEQ ID NO: 3; and wherein: an amino acid L at position 408 of SEQ ID NO: 3 is substituted with A, C, D, F, G, H, K, M, N, Q, S, W, or Y, preferably Y; an amino acid Y at position 409 of SEQ ID NO: 3 remains unchanged or is substituted with A, C, D, G, N, S, T or V, preferably A; an amino acid P at position 410 of SEQ ID NO: 3 remains unchanged or is substituted with A, C, G, I, L, M, N, S, T or V, preferably G; and an amino acid A at position 485 of SEQ ID NO: 3 is substituted with C, D, E, F, G, H, I, K, L, M, N, P, Q, R, T, V, W or Y, preferably E or L.

In accordance with some embodiments, the representative RNA polymerase variant is derived from Thermococcus sp. (strain 9° N-7) DNA polymerase (9° N) having a wild-type amino acid sequence of SEQ ID NO: 4; and wherein: an amino acid L at position 408 of SEQ ID NO: 4 is substituted with A, C, D, F, G, H, K, M, N, Q, S, W, or Y, preferably Y; an amino acid Y at position 409 of SEQ ID NO: 4 remains unchanged or is substituted with A, C, D, G, N, S, T or V, preferably A; and an amino acid P at position 410 of SEQ ID NO: 4 remains unchanged or is substituted with A, C, G, I, L, M, N, S, T or V, preferably G.

In accordance with some embodiments, the representative RNA polymerase variant is derived from Thermococcus sp. (strain 9° N-7) DNA polymerase (9° N) having a wild-type amino acid sequence of SEQ ID NO: 4; and wherein: an amino acid L at position 408 of SEQ ID NO: 4 is substituted with A, C, D, F, G, H, K, M, N, Q, S, W, or Y, preferably Y; an amino acid Y at position 409 of SEQ ID NO: 4 remains unchanged or is substituted with A, C, D, G, N, S, T or V preferably A; an amino acid P at position 410 of SEQ ID NO: 4 remains unchanged or is substituted with A, C, G, I, L, M, N, S, T or V, preferably G; and an amino acid A at position 485 of SEQ ID NO: 4 is substituted with C, D, E, F, G, H, I, K, L, M, N, P, Q, R, T, V, W or Y, preferably E or L.

In accordance with some embodiments, the representative RNA polymerase variant is derived from Pyrococcus furiosus DNA polymerase (Pfu) having a wild-type amino acid sequence of SEQ ID NO: 5; and wherein: an amino acid L at position 409 of SEQ ID NO: 5 is substituted with A, C, D, F, G, H, K, M, N, Q, S, W, or Y, preferably Y; an amino acid Y at position 410 of SEQ ID NO: 5 remains unchanged or is substituted with A, C, D, G, N, S, T or V, preferably A; and an amino acid P at position 411 of SEQ ID NO: 5 remains unchanged or is substituted with A, C, G, I, L, M, N, S, T or V, preferably G.

In accordance with some embodiments, the representative RNA polymerase variant is derived from Pyrococcus furiosus DNA polymerase (Pfu) having a wild-type amino acid sequence of SEQ ID NO: 5; and wherein: an amino acid L at position 409 of SEQ ID NO: 5 is substituted with A, C, D, F, G, H, K, M, N, Q, S, W, or Y, preferably Y; an amino acid Y at position 410 of SEQ ID NO: 5 remains unchanged or is substituted with A, C, D, G, N, S, T or V, preferably A; an amino acid P at position 411 of SEQ ID NO: 5 remains unchanged or is substituted with A, C, G, I, L, M, N, S, T or V, preferably G; and an amino acid A at position 486 of SEQ ID NO: 5 is substituted with C, D, E, F, G, H, I, K, L, M, N, P, Q, R, T, V, W or Y, preferably E or L.

In accordance with some embodiments, the representative RNA polymerase variant is derived from Thermococcus litoralis DNA polymerase (Vent) having a wild-type amino acid sequence of SEQ ID NO: 6; and wherein: an amino acid L at position 411 of SEQ ID NO: 6 is substituted with A, C, D, F, G, H, K, M, N, Q, S, W, or Y, preferably Y; an amino acid Y at position 412 of SEQ ID NO: 6 remains unchanged or is substituted with A, C, D, G, N, S, T or V, preferably A; and an amino acid P at position 413 of SEQ ID NO: 6 remains unchanged or is substituted with A, C, G, I, L, M, N, S, T or V, preferably G.

In accordance with some embodiments, the representative RNA polymerase variant is derived from Thermococcus litoralis DNA polymerase (Vent) having a wild-type amino acid sequence of SEQ ID NO: 6; and wherein: an amino acid L at position 411 of SEQ ID NO: 6 is substituted with A, C, D, F, G, H, K, M, N, Q, S, W, or Y, preferably Y; an amino acid Y at position 412 of SEQ ID NO: 6 remains unchanged or is substituted with A, C, D, G, N, S, T or V, preferably A; an amino acid P at position 413 of SEQ ID NO: 6 remains unchanged or is substituted with A, C, G, I, L, M, N, S, T or V, preferably G; and an amino acid A at position 488 of SEQ ID NO: 6 is substituted with C, D, E, F, G, H, I, K, L, M, N, P, Q, R, T, V, W or Y, preferably E or L.

In accordance with some embodiments, the representative RNA polymerase variant is derived from Methanosarcina acetivorans DNA polymerase (Mac) having a wild-type amino acid sequence of SEQ ID NO: 7; and wherein: an amino acid L at position 485 of SEQ ID NO: 7 is substituted with A, C, D, F, G, H, K, M, N, Q, S, W, or Y, preferably Y; an amino acid Y at position 486 of SEQ ID NO: 7 remains unchanged or is substituted with A, C, D, G, N, S, T or V, preferably A; and an amino acid P at position 487 of SEQ ID NO: 7 remains unchanged or is substituted with A, C, G, I, L, M, N, S, T or V, preferably G.

In accordance with some embodiments, the representative RNA polymerase variant is derived from Methanosarcina acetivorans DNA polymerase (Mac) having a wild-type amino acid sequence of SEQ ID NO: 7; and wherein: an amino acid L at position 485 of SEQ ID NO: 7 is substituted with A, C, D, F, G, H, K, M, N, Q, S, W, or Y, preferably Y; an amino acid Y at position 486 of SEQ ID NO: 7 remains unchanged or is substituted with A, C, D, G, N, S, T or V, preferably A; an amino acid P at position 487 of SEQ ID NO: 7 remains unchanged or is substituted with A, C, G, I, L, M, N, S, T or V, preferably G; and an amino acid A at position 565 of SEQ ID NO: 7 is substituted with C, D, E, F, G, H, I, K, L, M, N, P, Q, R, T, V, W or Y, preferably E or L.

In accordance with some embodiments, the representative RNA polymerase variant is derived from Pyrobaculum islandicum DNA polymerase (Pis) having a wild-type amino acid sequence of SEQ ID NO: 8; and wherein: an amino acid M at position 426 of SEQ ID NO: 8 is substituted with A, C, D, F, G, H, K, N, Q, S, W, or Y, preferably Y; an amino acid Y at position 427 of SEQ ID NO: 8 remains unchanged or is substituted with A, C, D, G, N, S, T or V, preferably A; and an amino acid P at position 428 of SEQ ID NO: 8 remains unchanged or is substituted with A, C, G, I, L, M, N, S, T or V, preferably G.

In accordance with some embodiments, the representative RNA polymerase variant is derived from Pyrobaculum islandicum DNA polymerase (Pis) having a wild-type amino acid sequence of SEQ ID NO: 8; and wherein: an amino acid M at position 426 of SEQ ID NO: 8 is substituted with A, C, D, F, G, H, K, N, Q, S, W, or Y, preferably Y; an amino acid Y at position 427 of SEQ ID NO: 8 remains unchanged or is substituted with A, C, D, G, N, S, T or V, preferably A; an amino acid P at position 428 of SEQ ID NO: 8 remains unchanged or is substituted with A, C, G, I, L, M, N, S, T or V, preferably G; and an amino acid A at position 508 of SEQ ID NO: 8 is substituted with C, D, E, F, G, H, I, K, L, M, N, P, Q, R, T, V, W or Y, preferably E or L.

In accordance with some embodiments, the representative RNA polymerase variant is derived from Sulfolobus solfataricus DNA polymerase (Sso) having a wild-type amino acid sequence of SEQ ID NO: 9; and wherein: an amino acid L at position 518 of SEQ ID NO: 9 is substituted with A, C, D, F, G, H, K, M, N, Q, S, W, or Y, preferably Y; an amino acid Y at position 519 of SEQ ID NO: 9 remains unchanged or is substituted with A, C, D, G, N, S, T or V, preferably A; and an amino acid P at position 520 of SEQ ID NO: 9 remains unchanged or is substituted with A, C, G, I, L, M, N, S, T or V, preferably G.

In accordance with some embodiments, the representative RNA polymerase variant is derived from Sulfolobus solfataricus DNA polymerase (Sso) having a wild-type amino acid sequence of SEQ ID NO: 9; and wherein: an amino acid L at position 518 of SEQ ID NO: 9 is substituted with A, C, D, F, G, H, K, M, N, Q, S, W, or Y, preferably Y; an amino acid Y at position 519 of SEQ ID NO: 9 remains unchanged or is substituted with A, C, D, G, N, S, T or V, preferably A; an amino acid P at position 520 of SEQ ID NO: 9 remains unchanged or is substituted with A, C, G, I, L, M, N, S, T or V, preferably G; and an amino acid A at position 601 of SEQ ID NO: 9 is substituted with C, D, E, F, G, H, I, K, L, M, N, P, Q, R, T, V, W or Y, preferably E or L.

In accordance with some embodiments, the representative RNA polymerase variant exhibits an activity of synthesizing nucleic acids in a template-independent manner by adding at least one nucleotide selected from the group of naturally occurring nucleotide, nucleotide analogue, or a mixture thereof, to an extendible initiator.

In certain embodiments, the extendible initiator comprises a single-stranded oligonucleotide initiator, a blunt ended double-stranded oligonucleotide initiator, or a mixture thereof. In certain embodiments, the extendible initiator is a free form nucleic acid and can be reacted in a liquid phase.

In certain embodiments, the extendible initiator is immobilized on a solid support, wherein the solid support comprises a particle, bead, slide, array surface, membrane, flow cell, well, microwell, nano-well, chamber, microfluidic chamber, channel, microfluidic channel, or any other surfaces.

In certain embodiments, the at least one nucleotide comprises a ribose. Furthermore, the at least one nucleotide is linked with a detectable label, such as fluorophores, enzymes, radioactive phosphates, digoxygenin, or biotin.

In accordance with some embodiments, the representative RNA polymerase variant exhibits the template-independent nucleic acid synthesis activity at reaction temperatures ranging from 10° C. to 100° C. For example, the reaction temperature is between 10° C. and 20° C., between 20° C. and 30° C., between 30° C. and 40° C., between 40° C. and 50° C., between 50° C. and 60° C., between 60° C. and 70° C., between 70° C. and 80° C., between 80° C. and 90° C., between 90° C. and 95° C., between 95° C. and 100° C., or any reaction temperatures within a range defined by an upper limit of 15° C., 20° C., 25° C., 30° C., 35° C., 37° C., 40° C., 45° C., 50° C., 55° C., 60° C., 65° C., 70° C., 75° C., 80° C., 85° C., 90° C., 95° C., or 100° C. and an lower limit of 10° C., 15° C., 20° C., 25° C., 30° C., 35° C., 37° C., 40° C., 45° C., 50° C., 55° C., 60° C., 65° C., 70° C., 75° C., 80° C., 85° C., 90° C., or 95° C.

### Creation of Polymerase Variants

Various types of mutagenesis techniques are optionally used in the present disclosure, e.g., to modify polymerases to create the variants of the subject application, or using random or semi-random mutational approaches. In general, any available mutagenesis procedure can be used for making polymerase mutants. Such mutagenesis procedures optionally include selection of altered nucleic acids and polypeptides for one or more activity of interest. Procedures that can be used include, but are not limited to: the site-directed point mutagenesis, random point mutagenesis, in vitro or in vivo homologous recombination (DNA shuffling and combinatorial overlap PCR), mutagenesis using uracil containing templates, oligonucleotide-directed mutagenesis, phosphorothioate-modified DNA mutagenesis, mutagenesis using gapped duplex DNA, point mismatch repair, mutagenesis using repair-deficient host strains, restriction-selection and restriction-purification, deletion mutagenesis, mutagenesis by total gene synthesis, degenerate PCR, double-strand break repair, and many others known to skilled person.

### Kit for Performing Template-Independent Nucleic Acid Synthesis Reaction

The present invention also provides a kit that includes the RNA polymerase variant described herein, for performing de novo enzymatic nucleic acid synthesis reaction, comprising: an RNA polymerase variant as described above, wherein the RNA polymerase variant exhibits activity of synthesizing nucleic acids in a template-independent manner by adding at least one nucleotide selected from the group of naturally occurring nucleotide, nucleotide analogue, or a mixture thereof, to an extendible initiator, thereby synthesizing a desired nucleic acid sequence.

Optionally, other reagents such as buffers and solutions required for the RNA polymerase variant and nucleotide solution are also included. Instructions for use of the assembled or packaged components are also typically but not necessarily included.

### Methods and Uses of RNA Polymerase Variant

In some embodiments, the RNA polymerase variants described herein can be used to add natural ribonucleotides (rNTP), such as rATP, rUTP, rCTP and rGTP, to the 3′-hydroxyl (3′-OH) terminus of a single-stranded or a blunt-end, duplex nucleic acids initiator in a template-independent synthesis manner to produce polynucleotides with desired sequences.

In some embodiments, the RNA polymerase variants described herein can be used to add natural ribonucleotides (rNTP), such as rATP, rUTP, rCTP and rGTP, to the 3′-OH termini of arrays of clustered single-stranded or a blunt-end, duplex nucleic acids initiators, which are immobilized or physically confined, and separated on a solid support as described previously; and preferably, the solid support is made of glass and implemented in the form of silicon wafer. Thus, a multiplexing, parallel de novo nucleic acid synthesis can be performed to synthesize large numbers of various polynucleotides or nucleic acids with distinct sequences.

In certain embodiments, the RNA polymerase variants described herein can be used to incorporate the nucleotide conjugates (one of the types of nucleotide analogue defined previously) covalently linked with an enzyme, an antibody, a chemical group, such as a biotin, a desthiobiotin, or a fluorophore on the base, phosphate moiety, or pentose sugar of nucleotide, to the 3′-terminus of the nucleic acid initiator in a template-independent synthesis manner.

The incorporation of these nucleotide analogues into the nucleic acids by RNA polymerase variants during the nucleic acid synthesis concurrently add the desired component, such as an associated enzyme, an antibody, or a chemical group to the newly synthesized nucleic acids in a base-specific, site-specific, or sequence-specific manner. Common components used to label or generate nucleic acid probes and conjugates are known in the art, which include, but are not limited to, radiolabeled nucleotides and nucleotide analogues, modified linkers, such as a biotin, a thiol, an azido, or an amine group, fluorophores, enzymes, and antibodies.

Alternatively, in other embodiments, to label or generate nucleic acid probes, the post-synthetic modifications of nucleic acids can be achieved by covalently or non-covalently coupling with an enzyme, an antibody, a chemical group, or a fluorophore via a modified linker on the base, the phosphate moiety, or the pentose sugar of synthesis nucleotide. As a result, the desired component can be covalently or non-covalently associated with the specific base or connected to the 5′- or 3′-terminus of newly synthesized nucleic acids.

In some embodiments, the RNA polymerase variant-dependent incorporation of linker-modified nucleotide analogues may be used to facilitate the newly synthesized polynucleotides or nucleic acids to be attached, immobilized or physically confined on various solid surfaces. Retrospectively, in other embodiments, the newly synthesized sequence-specific nucleic acids with unique labels, tags, or fluorophores can be used in various nucleic acid-based molecular detections, which include, but are not limited to, the fluorescence in situ hybridization (FISH), TaqMan real-time PCR (RT-PCR), real-time fluorescence ligase chain reaction (RT-LCR), real-time fluorescence recombinase-polymerase amplification (RPA) assay, and real-time fluorescence loop-mediated isothermal amplification assay.

The present disclosure further provides a method for template-independent synthesis of an RNA oligonucleotide, comprising:
(a) providing an initiator oligonucleotide,
(b) providing an RNA polymerase variant; and
(c) combining the initiator oligonucleotide, the RNA polymerase variant and one or more nucleotides under conditions sufficient for the addition of at least one nucleotide to the 3′ end of the initiator oligonucleotide.

Once the one or more nucleotides are added to the initiator oligonucleotide, one or more additional nucleotides can be added subsequently in order to synthesize a desired RNA oligonucleotide. Therefore, in certain embodiments, the method further comprises adding one or more natural or modified nucleotides to the 3′ end of the resulting RNA oligonucleotide (i.e., the RNA oligonucleotide formed in step (c)) until a desired RNA sequence is obtained. In certain embodiments, the method further comprises: (d) repeating steps (a)-(c) until a desired RNA sequence is obtained.

In certain embodiments, step (c) is carried out in the presence of one or more additional enzymes. In certain embodiments, step (c) is carried out in the presence of a mixture of two or more different enzymes. The mixture of enzymes may comprise more than one distinct RNA polymerase variants (e.g., 2 or 3 RNA polymerase variants).

In certain embodiments, step (c) is carried out in the presence of one or more additional enzyme (i.e., auxiliary enzyme), such as specific phosphatases in addition to the RNA polymerase variant. In certain embodiments, step (c) is carried out in the presence of a yeast inorganic pyrophosphatase (PPi-ase) in addition to the RNA polymerase variant.

In certain embodiments, the reaction in step (c) is carried out in the presence of one or more additional additives. In certain embodiments, step (c) is carried out in the presence of a crowding agent. In certain embodiments, the crowing agent is polyethylene glycol (PEG) or Ficoll. In certain embodiments, the crowding agent is polyethylene glycol (PEG). In certain embodiments, step (c) is carried out in the presence of an RNase inhibitor. In certain embodiments, step (c) is carried out in the presence of a non-hydrolyzable nucleotide.

### EXAMPLES

### Example 1: Preparation of RNA Polymerase Variants

The gene synthesis approach and mutagenesis technique are adapted to create exemplary RNA polymerase variants according to the properties of conserved/consensus amino acids in the conserved and semi-conserved regions of selective PolBs, which are disclosed herein. For instance, the well-known site-directed mutagenesis approach is conducted to change the amino acid residues in the motif Exo I, motif Exo II, motif Exo III, motif A, motif B, and motif C regions of an exemplary wild-type PolB listed herein.

In some embodiments, the procedure for obtaining exemplary RNA polymerase variants is generally divided into three steps, including Step 1: Gene synthesis of wild-type PolB and its 3′ to 5′ exonuclease-deficient (Exo⁻) mutant, Step 2: Construction of the specific exemplary RNA polymerase variant in the desired region, and Step 3: Expression and purification of wild-type PolB, Exo⁻ mutant, and RNA polymerase variant. As described in more detail below, the techniques used in said procedure are well-known to those skilled in the art.

In Step 1, the codon-optimized gene fragment encoding the wild-type, intein-free PolB polymerase is synthesized by Genomics BioSci & Tech Co. (New Taipei City, Taiwan). The 3′ to 5′ exonuclease-deficient (designated as Exo) PolB polymerase is also provided by the same vendor. The superscript “Exo⁻” following an abbreviated name of any PolB listed herein means that the designated wild-type PolB has been modified to eliminate the intrinsic 3′ to 5′ exonuclease activity, which indicates said PolB is a 3′ to 5′ exonuclease-deficient PolB. Preferably, in the Examples of this disclosure, the Exo means a PolB mutant carrying combinatory mutations at the positions corresponding to D354 of SEQ ID NO: 1, which is substituted with an alanine residue (D354A), and E356 of SEQ ID NO: 1, which is also substituted with an alanine residue (E356A), respectively.

In Step 2, the synthetic wild-type and Exo PolB genes are respectively subcloned into the pET28b vector using the flanking NdeI and NotI restriction sites. The sequences of recombinant plasmids are confirmed by DNA sequencing. To create the RNA polymerase variant at the desired motif region of the PolB Exo protein backbone, the site-directed mutagenesis, is conducted. Briefly, the site-directed mutagenesis PCR is performed with the recombinant plasmids using the Q5 Site-directed Mutagenesis Kit from New England Biolabs (Ipswich, MA) to introduce the amino acid substitution. The products are first analyzed by 1% agarose gel to confirm the amplicon size and the rest of PCR reaction mixture is then treated with DpnI at 37° C. for an hour. The mixture is further incubated at 70° C. for 10 mins to inactivate the DpnI function. The DpnI-treated PCR reaction mixture is then purified by the Qiagen's QIAquick PCR Purification Kit (Whatman, MA). The purified DNA fragment is treated with the mixture of T4 PNK and T4 DNA ligase. The re-circularized PCR-amplified DNA is transformed back into the E. coli cells. The plasmid DNA was later extracted from the E. coli cells using the Qiagen Plasmid Mini Kit (Whatman, MA). The mutated sequences of the polymerase variants at the desired motif region, or regions, are confirmed by DNA sequencing.

In Step 3, E. coli Acella cells harboring the plasmid DNA carrying specific polymerase variant gene are grown in 2 L of LB medium supplemented with 0.5% glucose and 50 μg/ml carbenicillin at 37° C. When the cell density reaches an absorbance value at OD_{600 nm} around 0.6-0.8, an 1 mM of isopropyl β-D-1-thiogalactopyranoside (IPTG) is added to induce protein expression. Cells are grown for additional 4 hours at 37° C. and then harvested by centrifugation at 4° C. for 10 min at 7,000×g. Cell pellets are resuspended with buffer A [50 mm Tris-HCl (pH 7.5), 300 mM NaCl, 0.5 mM EDTA, 1 mM DTT, 5% (v/v) glycerol] containing 1 mM benzamidine hydrochloride. Cell lysis is achieved by incubation with 50 mg of lysozyme on ice for 1 hour followed by sonication. The cell lysate is clarified by centrifugation at 18,000×g for 25 min at 4° C. The clarified crude cell extract is incubated at 70° C. for 30 minutes and then cooled down at 4° C. The heat-treated cell extract is further clarified by centrifugation at 18,000×g for 25 minutes at 4° C. After centrifugation, the supernatant is diluted with buffer A without NaCl and loaded onto a HiTrap Heparin column (Cytiva Life Sciences, Marlborough, MA, USA) pre-equilibrated in buffer A in the AKTA pure chromatography system (Cytiva Life Sciences, Marlborough, MA, USA). The protein is eluted with the linear 100 mM to 1 M NaCl gradient using the buffer B [50 mM Tris-HCl (pH 8.0), 1 M NaCl, 0.5 mM EDTA, 1 mM DTT, 5% (v/v) glycerol]. Column fractions are analyzed by 10% SDS-PAGE. Fractions containing desired protein are pooled and dialyzed against the storage buffer [50 mM Tris-HCl (pH 7.5), 250 mM NaCl, 0.5 mM EDTA, 1 mM DTT, 5% (v/v) glycerol] at 4° C. overnight. The dialyzed protein fraction pool containing the target protein is concentrated using an Amicon filter unit (MW cut-off 50,000). The concentrated protein pool is aliquoted and stored at −20° C. Each mutant polymerase variant was purified with the same procedures as described above. The final protein concentration is determined by the Bradford reaction (Bradford, 1976) using the Bio-Rad Protein Assay (Hercules, CA) with bovine serum albumin as a standard.

In this example, selected (exemplary) Exo RNA polymerase variants aimed for the following assays with functionally or positionally substitutions residing in the motif A and/or motif B are summarized and listed in Table 1

TABLE 1
List of amino acid substitutions in the exemplary RNA polymerase variants

| | | Equivalent substitutions in the motif A and/or motif B corresponding | | | |
|---|---|---|---|---|---|
| | | to the consensus sequence (SEQ ID NO: 1) | | | |
| Type of PolB | SEQ ID | Position 715 | Position 716 | Position 717 | Position 854 |
| Enzymes | NO | (motif A) | (motif A) | (motif A) | (motif B) |
| Tgo | 2 | L408A, L408C, | Y409A, Y409C, | P410A, P410C, | A485C, A485D, |
| | | L408D, L408F, | Y409D, Y409G, | P410G, P410I, | A485E, A485F, |
| | | L408G, L408H, | Y409N, Y409S, | P410L, P410M, | A485G, A485H, |
| | | L408K, L408M, | Y409T, Y409V | P410N, P410S, | A485I, A485K, |
| | | L408N, L408Q, | | P410T, P410V | A485L, A485M, |
| | | L408S, L408W, | | | A485N, A485P, |
| | | L408Y | | | A485Q, A485R, |
| | | | | | A485T, A485V, |
| | | | | | A485W, A485Y |
| Kod1 | 3 | L408A, L408C, | Y409A, Y409C, | P410A, P410C, | A485C, A485D, |
| | | L408D, L408F, | Y409D, Y409G, | P410G, P410I, | A485E, A485F, |
| | | L408G, L408H, | Y409N, Y409S, | P410L, P410M, | A485G, A485H, |
| | | L408K, L408M, | Y409T, Y409V | P410N, P410S, | A485I, A485K, |
| | | L408N, L408Q, | | P410T, P410V | A485L, A485M, |
| | | L408S, L408W, | | | A485N, A485P, |
| | | L408Y | | | A485Q, A485R, |
| | | | | | A485T, A485V, |
| | | | | | A485W, A485Y |
| 9°N | 4 | L408A, L408C, | Y409A, Y409C, | P410A, P410C, | A485C, A485D, |
| | | L408D, L408F, | Y409D, Y409G, | P410G, P410I, | A485E, A485F, |
| | | L408G, L408H, | Y409N, Y409S, | P410L, P410M, | A485G, A485H, |
| | | L408K, L408M, | Y409T, Y409V | P410N, P410S, | A485I, A485K, |
| | | L408N, L408Q, | | P410T, P410V | A485L, A485M, |
| | | L408S, L408W, | | | A485N, A485P, |
| | | L408Y | | | A485Q, A485R, |
| | | | | | A485T, A485V, |
| | | | | | A485W, A485Y |
| Pfu | 5 | L409A, L409C, | Y410A, Y410C, | P411A, P411C, | A486C, A486D, |
| | | L409D, L409F, | Y410D, Y410G, | P411G, P411I, | A486E, A486F, |
| | | L409G, L409H, | Y410N, Y410S, | P411L, P411M, | A486G, A486H, |
| | | L409K, L409M, | Y410T, Y410V | P411N, P411S, | A486I, A486K, |
| | | L409N, L409Q, | | P411T, P411V | A486L, A486M, |
| | | L409S, L409W, | | | A486N, A486P, |
| | | L409Y | | | A486Q, A486R, |
| | | | | | A486T, A486V, |
| | | | | | A486W, A486Y |
| Vent | 6 | L411A, LA11C, | Y412A, Y412C, | P413A, P413C, | A488C, A488D, |
| | | L411D, L411F, | Y412D, Y412G, | P413G, P413I, | A488E, A488F, |
| | | L411G, L411H, | Y412N, Y412S, | P413L, P413M, | A488G, A488H, |
| | | L411K, L411M, | Y412T, Y412V | P413N, P413S, | A488I, A488K, |
| | | L411N, L411Q, | | P413T, P413V | A488L, A488M, |
| | | L411S, L411W, | | | A488N, A488P, |
| | | L411Y | | | A488Q, A488R, |
| | | | | | A488T, A488V, |
| | | | | | A488W, A488Y |
| Mac | 7 | L485A, L485C, | Y486A, Y486C, | P487A, P487C, | A565C, A565D, |
| | | L485D, L485F, | Y486D, Y486G, | P487G, P487I, | A565E, A565F, |
| | | L485G, L485H, | Y486N, Y486S, | P487L, P487M, | A565G, A565H, |
| | | L485K, L485M, | Y486T, Y486V | P487N, P487S, | A565I, A565K, |
| | | L485N, L485Q, | | P487T, P487V | A565L, A565M, |
| | | L485S, L485W, | | | A565N, A565P, |
| | | L485Y | | | A565Q, A565R, |
| | | | | | A565T, A565V, |
| | | | | | A565W, A565Y |
| Pis | 8 | M426A, | Y427A, Y427C, | P428A, P428C, | A508C, A508D, |
| | | M426C, | Y427D, Y427G, | P428G, P428I, | A508E, A508F, |
| | | M426D, M426F, | Y427N, Y427S, | P428L, P428M, | A508G, A508H, |
| | | M426G, | Y427T, Y427V | P428N, P428S, | A508I, A508K, |
| | | M426H, | | P428T, P428V | A508L, A508M, |
| | | M426K, | | | A508N, A508P, |
| | | M426M, | | | A508Q, A508R, |
| | | M426N, | | | A508T, A508V, |
| | | M426Q, M426S, | | | A508W, A508Y |
| | | M426W, | | | |
| | | M426Y | | | |
| Sso | 9 | L518A, L518C, | Y519A, Y519C, | P520A, P520C, | A601C, A601D, |
| | | L518D, L518F, | Y519D, Y519G, | P520G, P520I, | A601E, A601F, |
| | | L518G, L518H, | Y519N, Y519S, | P520L, P520M, | A601G, A601H, |
| | | L518K, L518M, | Y519T, Y519V | P520N, P520S, | A601I, A601K, |
| | | L518N, L518Q, | | P520T, P520V | A601L, A601M, |
| | | L518S, L518W, | | | A601N, A601P, |
| | | L518Y | | | A601Q, A601R, |
| | | | | | A601T, A601V, |
| | | | | | A601W, A601Y |

### Example 2: Template-Independent RNA Synthesis Assay

The RNA polymerase variants provided herein are evaluated for template-independent RNA synthesis approach. To further determine the activities (performance on incorporating naturally occurring ribonucleotides) of the RNA polymerase variants, normal rNTPs and a single-stranded DNA initiator or a blunt-end duplex DNA initiator are used herein. Besides, the reaction temperature of said approach is set up as 55° C. to evaluate the heat-resistance of the exemplary RNA polymerase variants.

In this example, the following synthetic oligonucleotides are used as the initiators to determine the template-independent RNA synthesis activity of RNA polymerase variants.

FAM-45-mer ssDNA initiator for Mode I assay: a single-stranded DNA having the sequence of 5′-CTCGGCCTGGCACAGGTCCGTTCAGTGCTGCGGCGACCACCGAGG-3′ (SEQ ID NO: 18). This single-stranded oligonucleotide is labeled with a fluorescent fluorescein amidite (FAM) dye at the 5′-end.

Blunt-end duplex DNA initiator for Mode II assay: a duplex DNA composed of the FAM-45-mer ssDNA initiator pre-annealed with its complementary 45-mer oligonucleotide.

The blunt-end duplex DNA initiator is formed by annealing the FAM-45-mer ssDNA initiator primer with the complementary 45-mer DNA at a molar ratio of 1:1.5 in the 1×TE buffer [10 mM Tris-HCl (pH 8.0) and 1 mM EDTA] containing 100 mM NaCl. The DNA annealing reaction is performed in the Bio-Rad Thermal Cycler (Hercules, CA) by first heating up the sample mixture to 98° C. for 3 minute and then gradually cooling it down (5° C./30 seconds) to 4° C. The annealing product without overhang is used as the blunt-end duplex DNA initiator.

The template-independent RNA synthesis reaction is performed in the reaction mixtures (10 μl) containing 100 nM FAM-45-mer ssDNA initiator (Mode I assay) or the blunt-end duplex DNA initiator (Mode II assay), 0.25 mM manganese chloride (MnCl₂), and 200 nM exemplary RNA polymerase variant. The de novo enzymatic RNA synthesis reactions was initiated by the addition of 100 μM of rNTPs. The reactions were allowed to proceed for a defined period of time (e.g., 2 minutes for the Mode I assay and 10 minutes for the Mode II assay) and then terminated by adding 10 μl of 2× quench solution (95% de-ionized formamide and 25 mM EDTA) at a selected reaction temperature (.e.g., 55° C. for either the Mode I or Mode II assay). After either the reaction of Mode I or Mode II assay, the sample mixtures were first denatured at 95° C. for 10 min and analyzed by 20% polyacrylamide gel electrophoresis containing 8M urea (Urea-PAGE). The de novo enzymatic RNA synthesis reaction products are then visualized by imaging the gel on the Amersham Typhoon Laser Scanner (Cytiva Life Sciences, Marlborough, MA, USA).

Based on the assays described above, the relative template-independent RNA synthesis activity of each variant is scored and represented by the number of symbol “+”. The overall activity score for each variant is divided into 4 distinct levels: 1) the “+++” indicates that the initiator is completely elongated to various lengths of newly synthesized RNA as compared to the band intensity and position of the substrate control. Hence, the variant is considered to possess an 100% of RNA synthesis activity; 2) the “++” indicates that the initiator is elongated around 50% to 100% to various lengths of newly synthesized RNA as compared to the band intensity and position of the substrate control. Hence, the variant is considered to possess a 50% to an 100% of RNA synthesis activity; 3) the “+” indicates that the initiator is elongated around 10% to 50% to various lengths of newly synthesized RNA as compared to the band intensity and position of the substrate control. Hence, the variant is considered to possess a 10% to 50% of RNA synthesis activity; and 4) the “+/−” indicates that the initiator is elongated less than 10% to various lengths of newly synthesized RNA as compared to the band intensity and position of the substrate control. Therefore, the variant is considered to possess <10% of RNA synthesis activity. This principle for activity scoring is applied throughout the present disclosure and resulting data are primarily listed in the corresponding tables.

### Example 3: Catalytic Activity of RNA Polymerase Variants on Incorporating rNTPs to the FAM-45-Mer ssDNA Initiator and Blunt-End Duplex DNA Initiator

In this section, the selected exonuclease-deficient RNA polymerase variants (e.g., Tgo^{exo-}, Kod1^{exo-}, 9°N^{exo-}, Pfu^{exo-}, Vent^{exo-}, Mac^{exo-} and Sso^{exo-}) were modified to include on or more amino acid substitutions with different amino acids in varied conserved regions or motifs of each protein.

Additionally, in the preliminary screening, the inventor has discovered that a conserved motif of those exonuclease-deficient RNA polymerase variants, which is functionally and positionally equivalent to the L715, Y716, and P717 residing in the motif A of the consensus sequence (SEQ ID NO: 1) as defined herein, may be predominantly related with the function of de novo RNA synthesis. More specifically, said conserved motif may be an essential site for template-independent RNA synthesis activity. Furthermore, a conserved residue residing in the motif B, which is functionally and positionally equivalent to the A854 of the consensus sequence (SEQ ID NO: 1), serves as a reinforceable site for enhancing the template-independent RNA synthesis activity (data not shown).

### Example 3.1: Template-Independent RNA Synthesis Activity of Vent Variants

In this example, the RNA polymerase variants derived from Vent (SEQ ID NO: 6) is used exemplarily for evaluating the template-independent RNA synthesis activity of the variants carrying combinatory substitutions in the motif Exo I, the motif A, and the motif B. Additionally, the United States Patent NO. U.S. Ser. No. 11/136,564B2 disclosed an AAI motif for substituting said conserved motif of some archaeal DNA polymerases to improve the incorporation of nucleotide analogues for template-dependent DNA synthesis reactions (i.e., DNA sequencing). The conserved motif AAI is functionally and positionally equivalent to the L715, Y716, and P717 of the consensus sequence (SEQ ID NO: 1), therefore, the conserved motif is also functionally and positionally equivalent to the L411, Y412 and P413 residing in the motif A of the wild-type Vent (SEQ ID NO: 6). Thus, in view of the effects of the AAI motif on the template-directed nucleotide incorporation, the AAI motif substitution is equivalently included in this example for comparison. Moreover, in this example, the combinatory effect of substitution of A854 residing in the motif B of the consensus sequence, which is functionally and positionally equivalent to the A488 residing in the motif B of the wild-type Vent, is also evaluated.

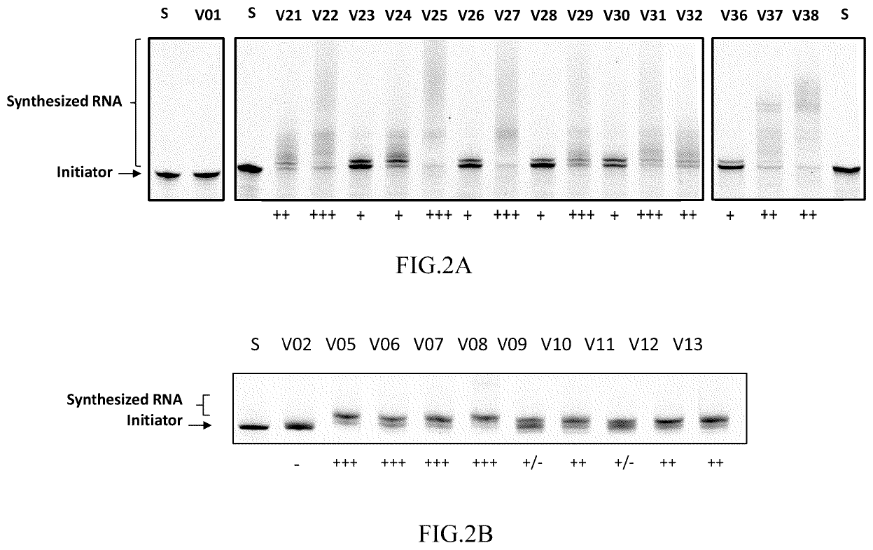

In this example, the variants modified from Vent Exo backbone are evaluated using the Mode I and Mode II activity assays as described above. The results of Mode I assay are shown in Table 2.1 and FIG. 2A; and the results of Mode II assay are shown in Table 2.2 and FIG. 2B, where “S” denoted in the figures stands for the substrate (FAM-45-mer ssDNA initiator or blunt-end duplex DNA initiator) and serves as a blank DNA control. Besides, for the sake of brevity, only the exemplary results for the representative Vent variants are shown in FIGS. 2A and 2B.

TABLE 2.1
List of amino acid substitutions and RNA synthesis activity scorings
of the exemplary Vent variants determined by Mode I activity assay

| Variant Name | Substitutions | Activity Scoring |
|---|---|---|
| V01 | D141A + E143A | − |
| V02 | D141A + E143A + A488L | − |
| V03 | D141A + E143A + P413E | +/− |
| V04 | D141A + E143A + L411Y | +/− |
| V05 | D141A + E143A + L411A + A488L | +/− |
| V06 | D141A + E143A + L411C + A488L | +/− |
| V07 | D141A + E143A + L411D + A488L | +/− |
| V08 | D141A + E143A + L411F + A488L | ++ |
| V09 | D141A + E143A + L411G + A488L | +/− |
| V10 | D141A + E143A + L411H + A488L | ++ |
| V11 | D141A + E143A + L411K + A488L | +/− |
| V12 | D141A + E143A + L411M + A488L | +/− |
| V13 | D141A + E143A + L411Q + A488L | +/− |
| V14 | D141A + E143A + L411Y + A488L | ++ |
| V15 | D141A + E143A + Y412A + A488L | + |
| V16 | D141A + E143A + Y412C + A488L | +/− |
| V17 | D141A + E143A + Y412G + A488L | + |
| V18 | D141A + E143A + Y412N + A488L | +/− |
| V19 | D141A + E143A + Y412S + A488L | + |
| V20 | D141A + E143A + P413S + A488L | ++ |
| V21 | D141A + E143A + L411A + Y412A + P413G + A488L | ++ |
| V22 | D141A + E143A + L411C + Y412A + P413G + A488L | +++ |
| V23 | D141A + E143A + L411D + Y412A + P413G + A488L | + |
| V24 | D141A + E143A + L411E + Y412A + P413G + A488L | + |
| V25 | D141A + E143A + L411F + Y412A + P413G + A488L | +++ |
| V26 | D141A + E143A + L411G + Y412A + P413G + A488L | + |
| V27 | D141A + E143A + L411H + Y412A + P413G + A488L | +++ |
| V28 | D141A + E143A + L411I + Y412A + P413G + A488L | + |
| V29 | D141A + E143A + L411K + Y412A + P413G + A488L | +++ |
| V30 | D141A + E143A + Y412A + P413G + A488L | + |
| V31 | D141A + E143A + L411M + Y412A + P413G + A488L | +++ |
| V32 | D141A + E143A + L411N + Y412A + P413G + A488L | ++ |
| V33 | D141A + E143A + L411Q + Y412A + P413G + A488L | +++ |
| V34 | D141A + E143A + L411S + Y412A + P413G + A488L | ++ |
| V35 | D141A + E143A + L411T + Y412A + P413G + A488L | + |
| V36 | D141A + E143A + L411V + Y412A + P413G + A488L | + |
| V37 | D141A + E143A + L411W + Y412A + P413G + A488L | ++ |
| V38 | D141A + E143A + L411Y + Y412A + P413G + A488L | ++ |
| V39 | D141A + E143A + L411Y + Y412C + P413G + A488L | + |
| V40 | D141A + E143A + L411Y + Y412D + P413G + A488L | ++ |
| V41 | D141A + E143A + L411Y + Y412F + P413G + A488L | + |
| V42 | D141A + E143A + L411Y + Y412G + P413G + A488L | ++ |
| V43 | D141A + E143A + L411Y + Y412H + P413G + A488L | + |
| V44 | D141A + E143A + L411Y + Y412I + P413G + A488L | + |
| V45 | D141A + E143A + L411Y + Y412L + P413G + A488L | + |
| V46 | D141A + E143A + L411Y + Y412M + P413G + A488L | + |
| V47 | D141A + E143A + L411Y + Y412N + P413G + A488L | ++ |
| V48 | D141A + E143A + L411Y + Y412Q + P413G + A488L | + |
| V49 | D141A + E143A + L411Y + Y412S + P413G + A488L | ++ |
| V50 | D141A + E143A + L411Y + Y412T + P413G + A488L | ++ |
| V51 | D141A + E143A + L411Y + Y412V + P413G + A488L | ++ |
| V52 | D141A + E143A + L411Y + P413G + A488L | ++ |
| V53 | D141A + E143A + L411Y + Y412A + P413A + A488L | ++ |
| V54 | D141A + E143A + L411Y + Y412A + P413C + A488L | ++ |
| V55 | D141A + E143A + L411Y + Y412A + P413D + A488L | + |
| V56 | D141A + E143A + L411Y + Y412A + P413E + A488L | + |
| V57 | D141A + E143A + L411Y + Y412A + P413F + A488L | + |
| V58 | D141A + E143A + L411Y + Y412A + P413H + A488L | + |
| V59 | D141A + E143A + L411Y + Y412A + P413I + A488L | ++ |
| V60 | D141A + E143A + L411Y + Y412A + P413K + A488L | + |
| V61 | D141A + E143A + L411Y + Y412A + P413L + A488L | ++ |
| V62 | D141A + E143A + L411Y + Y412A + P413M + A488L | ++ |
| V63 | D141A + E143A + L411Y + Y412A + P413N + A488L | ++ |
| V64 | D141A + E143A + L411Y + Y412A + A488L | ++ |
| V65 | D141A + E143A + L411Y + Y412A + P413Q + A488L | + |
| V66 | D141A + E143A + L411Y + Y412A + P413S + A488L | ++ |
| V67 | D141A + E143A + L411Y + Y412A + P413T + A488L | ++ |
| V68 | D141A + E143A + L411Y + Y412A + P413V + A488L | ++ |
| V69 | D141A + E143A + L411Y + Y412A + P413W + A488L | + |
| V70 | D141A + E143A + L411Y + Y412A + P413Y + A488L | + |
| V71 | D141A + E143A + L411Y + Y412A + P413G + A488A | + |
| V72 | D141A + E143A + L411Y + Y412A + P413G + A488C | ++ |
| V73 | D141A + E143A + L411Y + Y412A + P413G + A488D | ++ |
| V74 | D141A + E143A + L411Y + Y412A + P413G + A488E | ++ |
| V75 | D141A + E143A + L411Y + Y412A + P413G + A488F | ++ |
| V76 | D141A + E143A + L411Y + Y412A + P413G + A488G | ++ |
| V77 | D141A + E143A + L411Y + Y412A + P413G + A488H | ++ |
| V78 | D141A + E143A + L411Y + Y412A + P413G + A488I | ++ |
| V79 | D141A + E143A + L411Y + Y412A + P413G + A488K | ++ |
| V80 | D141A + E143A + L411Y + Y412A + P413G + A488M | ++ |
| V81 | D141A + E143A + L411Y + Y412A + P413G + A488N | ++ |
| V82 | D141A + E143A + L411Y + Y412A + P413G + A488P | + |
| V83 | D141A + E143A + L411Y + Y412A + P413G + A488Q | ++ |
| V84 | D141A + E143A + L411Y + Y412A + P413G + A488R | ++ |
| V85 | D141A + E143A + L411Y + Y412A + P413G + A488T | ++ |
| V86 | D141A + E143A + L411Y + Y412A + P413G + A488V | ++ |
| V87 | D141A + E143A + L411Y + Y412A + P413G + A488W | ++ |
| V88 | D141A + E143A + L411Y + Y412A + P413G + A488Y | ++ |

TABLE 2.2
List of amino acid substitutions and RNA synthesis activity scorings
of the exemplary Vent variants determined by Mode II activity assay

| Variant Name | Substitutions | Activity Scoring |
|---|---|---|
| V02 | D141A + E143A + A488L | − |
| V05 | D141A + E143A + L411A + A488L | +++ |
| V06 | D141A + E143A + L411C + A488L | +++ |
| V07 | D141A + E143A + L411D + A488L | +++ |
| V08 | D141A + E143A + L411F + A488L | +++ |
| V09 | D141A + E143A + L411G + A488L | −/+ |
| V10 | D141A + E143A + L411H + A488L | ++ |
| V11 | D141A + E143A + L411K + A488L | −/+ |
| V12 | D141A + E143A + L411M + A488L | ++ |
| V13 | D141A + E143A + L411Q + A488L | ++ |
| V14 | D141A + E143A + L411Y + A488L | +++ |
| V16 | D141A + E143A + Y412C + A488L | +++ |
| V17 | D141A + E143A + Y412G + A488L | +++ |
| V18 | D141A + E143A + Y412N + A488L | +++ |
| V19 | D141A + E143A + Y412S + A488L | +++ |
| V20 | D141A + E143A + P413S + A488L | +++ |
| V38 | D141A + E143A + L411Y + Y412A + P413G + A488L | +++ |
| V21 | D141A + E143A + L411A + Y412A + P413G + A488L | +++ |
| V22 | D141A + E143A + L411C + Y412A + P413G + A488L | +++ |
| V23 | D141A + E143A + L411D + Y412A + P413G + A488L | +++ |
| V24 | D141A + E143A + L411E + Y412A + P413G + A488L | +++ |
| V25 | D141A + E143A + L411F + Y412A + P413G + A488L | +++ |
| V26 | D141A + E143A + L411G + Y412A + P413G + A488L | +++ |
| V27 | D141A + E143A + L411H + Y412A + P413G + A488L | +++ |
| V28 | D141A + E143A + L411I + Y412A + P413G + A488L | +++ |
| V29 | D141A + E143A + L411K + Y412A + P413G + A488L | +++ |
| V30 | D141A + E143A + Y412A + P413G + A488L | +++ |
| V31 | D141A + E143A + L411M + Y412A + P413G + A488L | +++ |
| V32 | D141A + E143A + L411N + Y412A + P413G + A488L | +++ |
| V33 | D141A + E143A + L411Q + Y412A + P413G + A488L | +++ |
| V34 | D141A + E143A + L411S + Y412A + P413G + A488L | +++ |
| V35 | D141A + E143A + L411T + Y412A + P413G + A488L | +++ |
| V36 | D141A + E143A + L411V + Y412A + P413G + A488L | +++ |
| V37 | D141A + E143A + L411W + Y412A + P413G + A488L | +++ |
| V52 | D141A + E143A + L411Y + P413G + A488L | +++ |
| V39 | D141A + E143A + LA11Y + Y412C + P413G + A488L | +++ |
| V40 | D141A + E143A + L411Y + Y412D + P413G + A488L | +++ |
| V41 | D141A + E143A + L411Y + Y412F + P413G + A488L | +++ |
| V43 | D141A + E143A + L411Y + Y412H + P413G + A488L | +++ |
| V44 | D141A + E143A + L411Y + Y412I + P413G + A488L | +++ |
| V45 | D141A + E143A + L411Y + Y412L + P413G + A488L | +++ |
| V46 | D141A + E143A + L411Y + Y412M + P413G + A488L | +++ |
| V47 | D141A + E143A + L411Y + Y412N + P413G + A488L | +++ |
| V48 | D141A + E143A + L411Y + Y412Q + P413G + A488L | +++ |
| V49 | D141A + E143A + L411Y + Y412S + P413G + A488L | +++ |
| V50 | D141A + E143A + L411Y + Y412T + P413G + A488L | +++ |
| V51 | D141A + E143A + L411Y + Y412V + P413G + A488L | +++ |
| V64 | D141A + E143A + L411Y + Y412A + A488L | +++ |
| V53 | D141A + E143A + L411Y + Y412A + P413A + A488L | +++ |
| V54 | D141A + E143A + L411Y + Y412A + P413C + A488L | +++ |
| V55 | D141A + E143A + LA11Y + Y412A + P413D + A488L | +++ |
| V56 | D141A + E143A + L411Y + Y412A + P413E + A488L | +++ |
| V57 | D141A + E143A + L411Y + Y412A + P413F + A488L | +++ |
| V58 | D141A + E143A + L411Y + Y412A + P413H + A488L | +++ |
| V59 | D141A + E143A + L411Y + Y412A + P413I + A488L | +++ |
| V60 | D141A + E143A + L411Y + Y412A + P413K + A488L | +++ |
| V61 | D141A + E143A + L411Y + Y412A + P413L + A488L | +++ |
| V62 | D141A + E143A + L411Y + Y412A + P413M + A488L | +++ |
| V63 | D141A + E143A + LA11Y + Y412A + P413N + A488L | +++ |
| V65 | D141A + E143A + L411Y + Y412A + P413Q + A488L | +++ |
| V66 | D141A + E143A + L411Y + Y412A + P413S + A488L | +++ |
| V67 | D141A + E143A + L411Y + Y412A + P413T + A488L | +++ |
| V68 | D141A + E143A + L411Y + Y412A + P413V + A488L | +++ |
| V69 | D141A + E143A + L411Y + Y412A + P413W + A488L | +++ |
| V70 | D141A + E143A + L411Y + Y412A + P413Y + A488L | +++ |
| V72 | D141A + E143A + L411Y + Y412A + P413G + A488C | +++ |
| V73 | D141A + E143A + L411Y + Y412A + P413G + A488D | +++ |
| V74 | D141A + E143A + L411Y + Y412A + P413G + A488E | +++ |
| V75 | D141A + E143A + L411Y + Y412A + P413G + A488F | +++ |
| V77 | D141A + E143A + L411Y + Y412A + P413G + A488H | +++ |
| V78 | D141A + E143A + L411Y + Y412A + P413G + A488I | +++ |
| V79 | D141A + E143A + L411Y + Y412A + P413G + A488K | +++ |
| V80 | D141A + E143A + L411Y + Y412A + P413G + A488M | +++ |
| V81 | D141A + E143A + L411Y + Y412A + P413G + A488N | +++ |
| V82 | D141A + E143A + L411Y + Y412A + P413G + A488P | +++ |
| V83 | D141A + E143A + L411Y + Y412A + P413G + A488Q | +++ |
| V84 | D141A + E143A + L411Y + Y412A + P413G + A488R | +++ |
| V85 | D141A + E143A + L411Y + Y412A + P413G + A488T | +++ |
| V86 | D141A + E143A + L411Y + Y412A + P413G + A488V | +++ |
| V87 | D141A + E143A + L411Y + Y412A + P413G + A488W | +++ |
| V88 | D141A + E143A + L411Y + Y412A + P413G + A488Y | +++ |

As shown in Table 2.1, Table 2.2, FIG. 2A, and FIG. 2B, the variants carrying amino acid substitutions in the motif Exo I, motif A and/or motif B (A488L) have exerted prominent catalytic activity of template-independent enzymatic RNA synthesis in both Mode I and Mode II assays. More specifically, the preferable substitution combinations occurs in the motif A, such as L411A+Y412A+P413G (AAG), L411C+Y412A+P413G (CAG), L411F+Y412A+P413G (FAG), L411H+Y412A+P413G (HAG), L411K+Y412A+P413G (KAG), L411M+Y412A+P413G (MAG), L411Q+Y412A+P413G (QAG), and L411Y+Y412A+P413G (YAG) for the catalytic activity of template-independent enzymatic RNA synthesis.

### Example 3.2: Template-Independent RNA Synthesis Activity of Pfu Variants

In this example, the RNA polymerase variants derived from Pfu (SEQ ID NO: 5) is used exemplarily for evaluating the template-independent RNA synthesis activity of the variants carrying combinatory substitutions in the motif Exo I, the motif A, and the motif B.

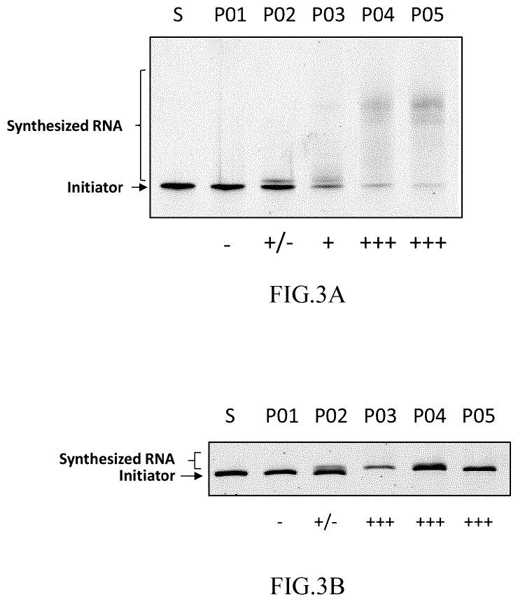

Specifically, the variants modified from Pfu Exo backbone are evaluated using the Mode I and Mode II activity assays as described above. The results of Mode I assay are shown in Table 3.1 and FIG. 3A; and the results of Mode II assay are shown in Table 3.2 and FIG. 3B, where “S” denoted in the figures stands for the substrate (FAM-45-mer ssDNA initiator or blunt-end duplex DNA initiator) and serves as a blank DNA control. Besides, for the sake of brevity, only the exemplary results for the representative Pfu variants are shown in this example.

TABLE 3.1
List of amino acid substitutions and RNA synthesis activity scorings
of the exemplary Pfu variants determined by Mode I activity assay

| Variant Name | Substitutions | Activity Scoring |
|---|---|---|
| P01 | D141A + E143A | − |
| P02 | D141A + E143A + A486L | +/− |
| P03 | D141A + E143A + L409Y + Y410A + P411G | + |
| P04 | D141A + E143A + L409Y + Y410A + P411G + A486L | +++ |
| P05 | D141A + E143A + L409A + Y410A + P411I + A486L | +++ |

TABLE 3.2
List of amino acid substitutions and RNA synthesis activity scorings
of the exemplary Pfu variants determined by Mode II activity assay

| Variant Name | Substitutions | Activity Scoring |
|---|---|---|
| P01 | D141A + E143A | − |
| P02 | D141A + E143A + A486L | +/− |
| P03 | D141A + E143A + L409Y + Y410A + P411G | +++ |
| P04 | D141A + E143A + L409Y + Y410A + P411G + A486L | +++ |
| P05 | D141A + E143A + L409A + Y410A + P411I + A486L | +++ |

As shown in FIG. 3A, FIG. 3B, Table 3.1 and Table 3.2, the variants carrying amino acid substitutions in the motif Exo I and motif A, such as variants P03, P04, and P05, have exerted prominent catalytic activity of template-independent enzymatic RNA synthesis in both Mode I and Mode II assays. Moreover, the variants carrying combinatory amino acid substitutions in the motif Exo I, motif A, and motif B, such as variants P04 and P05, further enhanced the said catalytic activity.

### Example 3.3: Template-Independent RNA Synthesis Activity of Kod1 Variants

In this example, the RNA polymerase variants derived from Kod1 (SEQ ID NO: 3) is used exemplarily for evaluating the template-independent RNA synthesis activity of the variants carrying combinatory substitutions in the motif Exo I, the motif A, and the motif B.

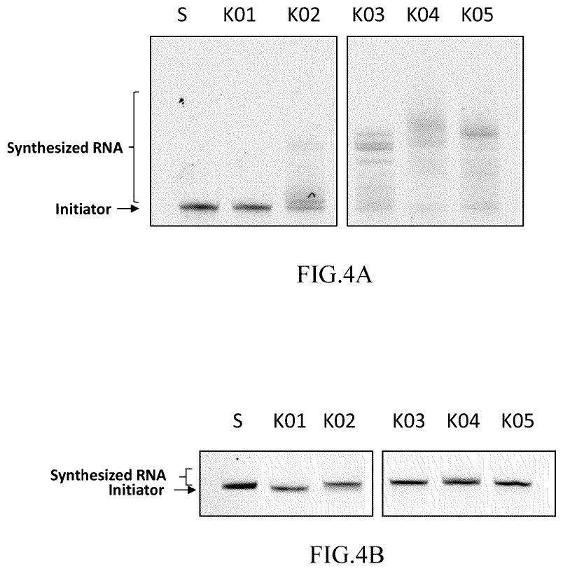

Specifically, the variants modified from Kod1 Exo⁻ backbone are evaluated using the Mode I and Mode II activity assays as described above. The results of Mode I assay are shown in Table 4.1 and FIG. 4A; and the results of Mode II assay are shown in Table 4.2 and FIG. 4B, where “S” denoted in the figures stands for the substrate (FAM-45-mer ssDNA initiator or blunt-end duplex DNA initiator) and serves as a blank DNA control. Besides, for the sake of brevity, only the exemplary results for the representative Kod1 variants are shown in this example.

TABLE 4.1
List of amino acid substitutions and RNA synthesis activity scorings
of the exemplary Kod1 variants determined by Mode I activity assay

| Variant Name | Substitutions | Activity Scoring |
|---|---|---|
| K01 | D141A + E143A | − |
| K02 | D141A + E143A + A485L | + |
| K03 | D141A + E143A + L408Y + Y409A + P410G | ++ |
| K04 | D141A + E143A + L408Y + Y409A + P410G + A485L | +++ |
| K05 | D141A + E143A + L408A + Y409A + P410I + A485L | ++ |

TABLE 4.2
List of amino acid substitutions and RNA synthesis activity scorings
of the exemplary Kod1 variants determined by Mode II activity assay

| Variant Name | Substitutions | Activity Scoring |
|---|---|---|
| K01 | D141A + E143A | − |
| K02 | D141A + E143A + A485L | + |
| K03 | D141A + E143A + L408Y + Y409A + P410G | +++ |
| K04 | D141A + E143A + L408Y + Y409A + P410G + A485L | +++ |
| K05 | D141A + E143A + L408A + Y409A + P410I + A485L | +++ |

As shown in FIG. 4A, FIG. 4B, Table 4.1 and Table 4.2, the variants carrying amino acid substitutions in the motif Exo I, motif A and/or motif B, such as variants K02, K03, K04, and K05, have exerted prominent catalytic activity of template-independent enzymatic RNA synthesis in both Mode I and Mode II assays. Moreover, the variants carrying combinatory amino acid substitutions in the motif Exo I, motif A, and motif B (A485L), such as K04 and K05, further enhanced the said catalytic activity.

### Example 3.4: Template-Independent RNA Synthesis Activity of Mac Variants

In this example, the RNA polymerase variants derived from Mac (SEQ ID NO: 7) is used exemplarily for evaluating the template-independent RNA synthesis activity of the variants carrying combinatory substitutions in the motif Exo I, the motif A, and the motif B.

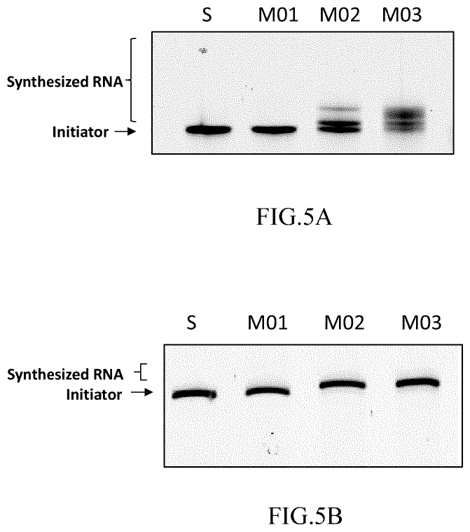

Specifically, the variants modified from Mac Exo⁻ backbone are evaluated using the Mode I and Mode II activity assays as described above. The results of Mode I assay are shown in Table 5.1 and FIG. 5A; and the results of Mode II assay are shown in Table 5.2 and FIG. 5B, where “S” denoted in the figures stands for the substrate (FAM-45-mer ssDNA initiator or blunt-end duplex DNA initiator) and serves as a blank DNA control. Besides, for the sake of brevity, only the exemplary results for the representative Mac variants are shown in this example.

TABLE 5.1
List of amino acid substitutions and RNA synthesis activity scorings
of the exemplary Mac variants determined by Mode I activity assay

| Variant Name | Substitutions | Activity Scoring |
|---|---|---|
| M01 | D198A + E200A | − |
| M02 | D198A + E200A + L485Y + Y486A + P487G | +/− |
| M03 | D198A + E200A + L485Y + Y486A + P487G + A565L | + |

TABLE 5.2
List of amino acid substitutions and RNA synthesis activity scorings
of the exemplary Mac variants determined by Mode II activity assay

| Variant Name | Substitutions | Activity Scoring |
|---|---|---|
| M01 | D198A + E200A | − |
| M02 | D198A + E200A + L485Y + Y486A + P487G | +++ |
| M03 | D198A + E200A + L485Y + Y486A + P487G + A565L | +++ |

As shown in FIG. 5A, FIG. 5B, Table 5.1 and Table 5.2, the variants carrying amino acid substitutions in the motif Exo I and motif A, such as variant M02, have exerted prominent catalytic activity of template-independent enzymatic RNA synthesis in both Mode I and Mode II assays. Moreover, the variants carrying combinatory amino acid substitutions in the motif Exo I, motif A, and motif B, such as variant M03, further enhanced the said catalytic activity.

### Example 3.5: Template-Independent RNA Synthesis Activity of Tgo Variants

In this example, the RNA polymerase variants derived from Tgo (SEQ ID NO: 2) is used exemplarily for evaluating the template-independent RNA synthesis activity the variants carrying combinatory substitutions in the motif Exo I, the motif A, and the motif B.

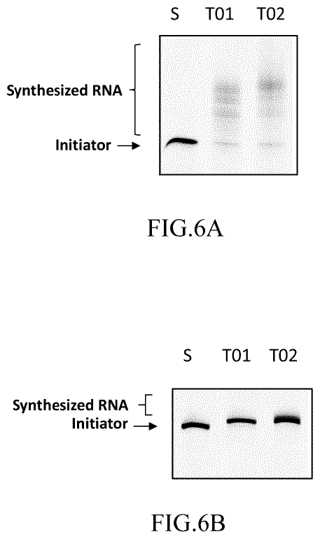

Specifically, the variants modified from Tgo Exo backbone are evaluated using the Mode I and Mode II activity assays as described above. The results of Mode I assay are shown in Table 6.1 and FIG. 6A; and the results of Mode II assay are shown in Table 6.2 and FIG. 6B, where “S” denoted in the figures stands for the substrate (FAM-45-mer ssDNA initiator or blunt-end duplex DNA initiator) and serves as a blank DNA control. Besides, for the sake of brevity, only the exemplary results for the representative Tgo variants are shown in this example.

TABLE 6.1
List of amino acid substitutions and RNA synthesis activity scorings
of the exemplary Tgo variants determined by Mode I activity assay

| Variant Name | Substitutions | Activity Scoring |
|---|---|---|
| T01 | D141A + E143A + L408A + Y409A + P410A + A485L | +++ |
| T02 | D141A + E143A + L408Y + Y409A + P410G + A485L | +++ |

TABLE 6.2
List of amino acid substitutions and RNA synthesis activity scorings
of the exemplary Tgo variants determined by Mode II activity assay

| Variant Name | Substitutions | Activity Scoring |
|---|---|---|
| T01 | D141A + E143A + LA08A + Y409A + P410A + A485L | +++ |
| T02 | D141A + E143A + L408Y + Y409A + P410G + A485L | +++ |

As shown in FIG. 6A, FIG. 6B, Table 6.1 and Table 6.2, the variants carrying amino acid substitutions in motif Exo I, motif A, and motif B, such as variants T01 and T02, have exerted prominent catalytic activity of template-independent enzymatic RNA synthesis in both Mode I and Mode II assays.

### Example 3.6: Template-Independent RNA Synthesis Activity of Sso Variants

In this example, the RNA polymerase variants derived from Sso (SEQ ID NO: 9) is used exemplarily for evaluating the template-independent RNA synthesis activity of the variants carrying combinatory substitutions in the motif Exo I, the motif A, and the motif B.

Specifically, the variants modified from Sso Exo backbone are evaluated using the Mode I and Mode II activity assays as described above. The results of Mode I and Mode II assays are shown in Tables 7.1 and 7.2 respectively. For the sake of brevity, the Urea-PAGE image results in this example is currently omitted.

TABLE 7.1
List of amino acid substitutions and activity scorings of the
exemplary Sso variants determined by Mode I activity assay

| Variant Name | Substitutions | Activity Scoring |
|---|---|---|
| S01 | D231A + E233A | − |
| S03 | D231A + E233A + L518Y + Y519A + P520G | +++ |
| S06 | D231A + E233A + L518Y + Y519A + P520G + A601L | +++ |

TABLE 7.2
List of amino acid substitutions and activity scorings of the
exemplary Sso variants determined by Mode II activity assay

| Variant Name | Substitutions | Activity Scoring |
|---|---|---|
| S01 | D231A + E233A | − |
| S03 | D231A + E233A + L518Y + Y519A + P520G | +++ |
| S06 | D231A + E233A + L518Y + Y519A + P520G + A601L | +++ |

As shown in the Tables 7.1 and 7.2, the variants carrying amino acid substitutions in motif Exo I, motif A, and/or motif B, such as variants S03 and S06, have exerted prominent catalytic activity of template-independent enzymatic RNA synthesis in both Mode I and Mode II assays.

### Example 3.7: Template-Independent RNA Synthesis Activity of 9° N Variants

In this example, the RNA polymerase variants derived from 9N (SEQ ID NO: 4) is used exemplarily for evaluating the template-independent RNA synthesis activity of the variants carrying combinatory substitutions in the motif Exo I, the motif A, and the motif B.

Specifically, the variants modified from 9° N Exo⁻ backbone are evaluated using the Mode I and Mode II activity assays as described above. The results of Mode I and Mode II assays are shown in Tables 8.1 and 8.2 respectively. For the sake of brevity, the Urea-PAGE image results in this example is currently omitted.

TABLE 8.1
List of amino acid substitutions and activity scorings of the
exemplary 9°N variants determined by Mode I activity assay

| Variant Name | Substitutions | Activity Scoring |
|---|---|---|
| N02 | D141A + E143A + L408Y + Y409A + P410E + A485V | ++ |
| N03 | D141A + E143A + L408Y + Y409A + P410F + A485V | ++ |
| N04 | D141A + E143A + L408Y + Y409A + P410G + A485V | +++ |
| N05 | D141A + E143A + L408Y + Y409A + P410H + A485V | +++ |
| N06 | D141A + E143A + L408Y + Y409A + P410T + A485V | +++ |
| N07 | D141A + E143A + L408Y + Y409A + P410V + A485V | +++ |
| N08 | D141A + E143A + L408Y + Y409C + P410G + A485V | ++ |
| N09 | D141A + E143A + L408Y + Y409G + P410G + A485V | ++ |
| N10 | D141A + E143A + L408Y + Y409I + P410G + A485V | ++ |
| N11 | D141A + E143A + L408Y + Y409K + P410G + A485V | +/− |
| N12 | D141A + E143A + L408Y + Y409L + P410G + A485V | +/− |
| N13 | D141A + E143A + LA08Y + Y409Q + P410G + A485V | +/− |
| N14 | D141A + E143A + L408Y + Y409Y + P410G + A485V | +++ |
| N15 | D141A + E143A + L408A + Y409A + P410G + A485V | ++ |
| N16 | D141A + E143A + L408S + Y409A + P410G + A485V | ++ |
| N17 | D141A + E143A + L408V + Y409A + P410G + A485V | +++ |

TABLE 8.2
List of amino acid substitutions and activity scorings of the
exemplary 9°N variants determined by Mode II activity assay

| Variant Name | Substitutions | Activity Scoring |
|---|---|---|
| N02 | D141A + E143A + LA08Y + Y409A + P410E + A485V | +++ |
| N03 | D141A + E143A + L408Y + Y409A + P410F + A485V | +++ |
| N04 | D141A + E143A + L408Y + Y409A + P410G + A485V | +++ |
| N05 | D141A + E143A + L408Y + Y409A + P410H + A485V | +++ |
| N06 | D141A + E143A + L408Y + Y409A + P410T + A485V | +++ |
| N07 | D141A + E143A + L408Y + Y409A + P410V + A485V | +++ |
| N08 | D141A + E143A + L408Y + Y409C + P410G + A485V | +++ |
| N09 | D141A + E143A + L408Y + Y409G + P410G + A485V | +++ |
| N10 | D141A + E143A + L408Y + Y409I + P410G + A485V | +++ |
| N11 | D141A + E143A + L408Y + Y409K + P410G + A485V | +++ |
| N12 | D141A + E143A + L408Y + Y409L + P410G + A485V | +++ |
| N13 | D141A + E143A + L408Y + Y409Q + P410G + A485V | +++ |
| N14 | D141A + E143A + LA08Y + Y409Y + P410G + A485V | +++ |
| N15 | D141A + E143A + L408A + Y409A + P410G + A485V | +++ |
| N16 | D141A + E143A + L408S + Y409A + P410G + A485V | +++ |
| N17 | D141A + E143A + L408V + Y409A + P410G + A485V | ++ |

As shown in the Tables 8.1 and 8.2, the variants carrying amino acid substitutions in motif Exo I, motif A, and motif B, such as the variants listed in Tables 8.1 and 8.2, have exerted prominent catalytic activity of template-independent enzymatic RNA synthesis in both Mode I and Mode II assays.

In view of the results observed, the RNA polymerase variants and the kit provided herein have been further proven in various scenarios to use rNTPs effectively and efficiently for de novo enzymatic RNA synthesis. Furthermore, these RNA polymerase variants are also proven to successfully exert the conferred template-independent RNA synthesis function under broader reaction temperatures covering from atmospheric temperatures to the hyperthermal conditions, demonstrating a higher thermotolerance. Therefore, the RNA polymerase variants and the kit within the scope of the present disclosure can broaden the scope of various applications of template-independent enzymatic nucleic acids synthesis in different reaction conditions.

The present disclosure has been described with embodiments thereof, and it is understood that various modifications, without departing from the scope of the present disclosure, are in accordance with the embodiments of the present disclosure. Hence, the embodiments described are intended to cover the modifications within the scope of the present disclosure, rather than to limit the present disclosure. The scope of the claims therefore should be accorded the broadest interpretation so as to encompass all such modifications.

What is claimed is:

1. An DNA polymerase variant having a template-independent RNA synthesis function and modified from a wild-type B-family DNA polymerase having an amino acid sequence selected from the group consisting of SEQ ID NOs: 2, 3, 4, 5, 6, 7, 8, 9, 10, 11, 12, 13, 14, 15, 16 and 17,
wherein:
the amino acid L at position 408 of SEQ ID NO: 2, 3, or 4 is substituted with A, C, D, F, G, H, K, M, N, Q, S, W, or Y;
the amino acid L at position 409 of SEQ ID NO: 5 is substituted with A, C, D, F, G, H, K, M, N, Q, S, W, or Y;
the amino acid L at position 411 of SEQ ID NO: 6 is substituted with A, C, D, F, G, H, K, M, N, Q, S, W, or Y;
the amino acid L at position 485 of SEQ ID NO: 7 is substituted with A, C, D, F, G, H, K, M, N, Q, S, W, or Y;
the amino acid M at position 426 of SEQ ID NO: 8 is substituted with A, C, D, F, G, H, K, M, N, Q, S, W, or Y;
the amino acid L at position 518 of SEQ ID NO: 9 is substituted with A, C, D, F, G, H, K, M, N, Q, S, W, or Y;
the amino acid L at position 417 of SEQ ID NO: 10 is substituted with A, C, D, F, G, H, K, M, N, Q, S, W, or Y;
an amino acid L at position 606 of SEQ ID NO: 11 is substituted with A, C, D, F, G, H, K, M, N, Q, S, W, or Y;
an amino acid L at position 612 of SEQ ID NO: 12 is substituted with A, C, D, F, G, H, K, M, N, Q, S, W, or Y;
an amino acid L at position 425 of SEQ ID NO: 13 is substituted with A, C, D, F, G, H, K, M, N, Q, S, W, or Y;
an amino acid L at position 423 of SEQ ID NO: 14 is substituted with A, C, D, F, G, H, K, M, N, Q, S, W, or Y;
an amino acid L at position 415 of SEQ ID NO: 15 is substituted with A, C, D, F, G, H, K, M, N, Q, S, W, or Y;
an amino acid L at position 412 of SEQ ID NO: 16 is substituted with A, C, D, F, G, H, K, M, N, Q, S, W, or Y; or
an amino acid L at position 253 of SEQ ID NO: 17 is substituted with A, C, D, F, G, H, K, M, N, Q, S, W, or Y, and
wherein the RNA polymerase variant has a reduced or deficient 3′ to 5′ exonuclease activity.

2. The DNA polymerase variant of claim 1, wherein the wild-type B-family DNA polymerase has the amino acid sequence selected from the group consisting of SEQ ID NO: 2, 3, 4, 5, 6, 7, 8 and 9.

3. The DNA polymerase variant of claim 2, wherein the wild-type B-family DNA polymerase is Thermococcus gorgonarius DNA polymerase (Tgo), Thermococcus kodakarensis DNA polymerase (Kod1), Thermococcus sp. (strain 9° N-7) DNA polymerase (9° N), Pyrococcus furiosus DNA polymerase (Pfu), Thermococcus litoralis DNA polymerase (Vent), Methanosarcina acetivorans DNA polymerase (Mac), Pyrobaculum islandicum DNA polymerase (Pis), Sulfolobus solfataricus DNA polymerase (Sso), Methanococcus maripaludis DNA polymerase (Mma), human DNA polymerase, delta catalytic p125 subunit (hPOLD), Saccharomyces cerevisiae DNA polymerase delta catalytic subunit (ScePOLD), Pseudomonas aeruginosa DNA polymerase II (Pae), Escherichia. coli DNA polymerase II (Eco), Escherichia coli phage RB69 DNA polymerase (RB69), Escherichia coli phage T4 DNA polymerase (T4), or Bacillus phage Phi29 DNA polymerase (Phi29).

4. The DNA polymerase variant of claim 1, wherein the DNA polymerase variant is derived from Thermococcus gorgonarius DNA polymerase (Tgo) having a wild-type amino acid sequence of SEQ ID NO: 2; and wherein:
the amino acid Y at position 409 of SEQ ID NO: 2 remains unchanged or is substituted with A, C, D, G, N, S, T or V; and
the amino acid P at position 410 of SEQ ID NO: 2 remains unchanged or is substituted with A, C, G, I, L, M, N, S, T or V.

5. The DNA polymerase variant of claim 1, wherein the DNA polymerase variant is derived from Thermococcus gorgonarius DNA polymerase (Tgo) having a wild-type amino acid sequence of SEQ ID NO: 2; and wherein:
the amino acid Y at position 409 of SEQ ID NO: 2 remains unchanged or is substituted with A, C, D, G, N, S, T or V;
the amino acid P at position 410 of SEQ ID NO: 2 remains unchanged or is substituted with A, C, G, I, L, M, N, S, T or V; and
the amino acid A at position 485 of SEQ ID NO: 2 is substituted with C, D, E, F, G, H, I, K, L, M, N, P, Q, R, T, V, W or Y.

6. The DNA polymerase variant of claim 1, wherein the DNA polymerase variant is derived from Thermococcus kodakarensis DNA polymerase (Kod1) having a wild-type amino acid sequence of SEQ ID NO: 3; and wherein:
the amino acid Y at position 409 of SEQ ID NO: 3 remains unchanged or is substituted with A, C, D, G, N, S, T or V; and
the amino acid P at position 410 of SEQ ID NO: 3 remains unchanged or is substituted with A, C, G, I, L, M, N, S, T or V.

7. The DNA polymerase variant of claim 1, wherein the DNA polymerase variant is derived from Thermococcus kodakarensis DNA polymerase (Kod1) having a wild-type amino acid sequence of SEQ ID NO: 3; and wherein:
i. the amino acid Y at position 409 of SEQ ID NO: 3 remains unchanged or is substituted with A, C, D, G, N, S, T or V;
ii. the amino acid P at position 410 of SEQ ID NO: 3 remains unchanged or is substituted with A, C, G, I, L, M, N, S, T or V; and
iii. the amino acid A at position 485 of SEQ ID NO: 3 is substituted with C, D, E, F, G, H, I, K, L, M, N, P, Q, R, T, V, W or Y.

8. The DNA polymerase variant of claim 1, wherein the DNA polymerase variant is derived from Thermococcus sp. (strain 9° N-7) DNA polymerase (9° N) having a wild-type amino acid sequence of SEQ ID NO: 4; and wherein:
the amino acid Y at position 409 of SEQ ID NO: 4 remains unchanged or is substituted with A, C, D, G, N, S, T or V; and
the amino acid P at position 410 of SEQ ID NO: 4 remains unchanged or is A, C, G, I, L, M, N, S, T or V.

9. The DNA polymerase variant of claim 1, wherein the DNA polymerase variant is derived from Thermococcus sp. (strain 9° N-7) DNA polymerase (9° N) having a wild-type amino acid sequence of SEQ ID NO: 4; and wherein:
the amino acid Y at position 409 of SEQ ID NO: 4 remains unchanged or is r substituted with A, C, D, G, N, S, T or V;
the amino acid P at position 410 of SEQ ID NO: 4 remains unchanged or is substituted with A, C, G, I, L, M, N, S, T or V; and
the amino acid A at position 485 of SEQ ID NO: 4 is substituted with C, D, E, F, G, H, I, K, L, M, N, P, Q, R, T, V, W or Y.

10. The DNA polymerase variant of claim 1, wherein the DNA polymerase variant is derived from Pyrococcus furiosus DNA polymerase (Pfu) having a wild-type amino acid sequence of SEQ ID NO: 5; and wherein:
the amino acid Y at position 410 of SEQ ID NO: 5 remains unchanged or is substituted with A, C, D, G, N, S, T or V; and
the amino acid P at position 411 of SEQ ID NO: 5 remains unchanged or is substituted with A, C, G, I, L, M, N, S, T or V.

11. The DNA polymerase variant of claim 1, wherein the DNA polymerase variant is derived from Pyrococcus furiosus DNA polymerase (Pfu) having a wild-type amino acid sequence of SEQ ID NO: 5; and wherein:
the amino acid Y at position 410 of SEQ ID NO: 5 remains unchanged or is substituted with A, C, D, G, N, S, T or V;
the amino acid P at position 411 of SEQ ID NO: 5 remains unchanged or is substituted with A, C, G, I, L, M, N, S, T or V; and
the amino acid A at position 486 of SEQ ID NO: 5 is substituted with C, D, E, F, G, H, I, K, L, M, N, P, Q, R, T, V, W or Y.

12. The DNA polymerase variant of claim 1, wherein the DNA polymerase variant is derived from Thermococcus litoralis DNA polymerase (Vent) having a wild-type amino acid sequence of SEQ ID NO: 6; and wherein:
the amino acid Y at position 412 of SEQ ID NO: 6 remains unchanged or is substituted with A, C, D, G, N, S, T or V; and
the amino acid P at position 413 of SEQ ID NO: 6 remains unchanged or is substituted with A, C, G, I, L, M, N, S, T or V.

13. The DNA polymerase variant of claim 1, wherein the DNA polymerase variant is derived from Thermococcus litoralis DNA polymerase (Vent) having a wild-type amino acid sequence of SEQ ID NO: 6; and wherein:
the amino acid Y at position 412 of SEQ ID NO: 6 remains unchanged or is substituted with A, C, D, G, N, S, T or V;
the amino acid P at position 413 of SEQ ID NO: 6 remains unchanged or is substituted with A, C, G, I, L, M, N, S, T or V; and
the amino acid A at position 488 of SEQ ID NO: 6 is substituted with C, D, E, F, G, H, I, K, L, M, N, P, Q, R, T, V, W or Y.

14. The DNA polymerase variant of claim 1, wherein the DNA polymerase variant is derived from Methanosarcina acetivorans DNA polymerase (Mac) having a wild-type amino acid sequence of SEQ ID NO: 7; and wherein:
the amino acid Y at position 486 of SEQ ID NO: 7 remains unchanged or is substituted with A, C, D, G, N, S, T or V; and
the amino acid P at position 487 of SEQ ID NO: 7 remains unchanged or is substituted with A, C, G, I, L, M, N, S, T or V.

15. The DNA polymerase variant of claim 1, wherein the DNA polymerase variant is derived from Methanosarcina acetivorans DNA polymerase (Mac) having a wild-type amino acid sequence of SEQ ID NO: 7; and wherein:
the amino acid Y at position 486 of SEQ ID NO: 7 remains unchanged or is substituted with A, C, D, G, N, S, T or V;
the amino acid P at position 487 of SEQ ID NO: 7 remains unchanged or is substituted with A, C, G, I, L, M, N, S, T or V; and
the amino acid A at position 565 of SEQ ID NO: 7 is substituted with C, D, E, F, G, H, I, K, L, M, N, P, Q, R, T, V, W or Y.

16. The DNA polymerase variant of claim 1, wherein the DNA polymerase variant is derived from Pyrobaculum islandicum DNA polymerase (Pis) having a wild-type amino acid sequence of SEQ ID NO: 8; and wherein:
the amino acid Y at position 427 of SEQ ID NO: 8 remains unchanged or is substituted with A, C, D, G, N, S, T or V; and
the amino acid P at position 428 of SEQ ID NO: 8 remains unchanged or is substituted with A, C, G, I, L, M, N, S, T or V.

17. The DNA polymerase variant of claim 1, wherein the DNA polymerase variant is derived from Pyrobaculum islandicum DNA polymerase (Pis) having a wild-type amino acid sequence of SEQ ID NO: 8; and wherein:
the amino acid Y at position 427 of SEQ ID NO: 8 remains unchanged or is substituted with A, C, D, G, N, S, T or V;
the amino acid P at position 428 of SEQ ID NO: 8 remains unchanged or is substituted with A, C, G, I, L, M, N, S, T or V; and
the amino acid A at position 508 of SEQ ID NO: 8 is substituted with C, D, E, F, G, H, I, K, L, M, N, P, Q, R, T, V, W or Y.

18. The DNA polymerase variant of claim 1, wherein the DNA polymerase variant is derived from Sulfolobus solfataricus DNA polymerase (Sso) having a wild-type amino acid sequence of SEQ ID NO: 9; and wherein:
the amino acid Y at position 519 of SEQ ID NO: 9 remains unchanged or is substituted with A, C, D, G, N, S, T or V; and
the amino acid P at position 520 of SEQ ID NO: 9 remains unchanged or is substituted with A, C, G, I, L, M, N, S, T or V.

19. The DNA polymerase variant of claim 1, wherein the DNA polymerase variant is derived from Sulfolobus solfataricus DNA polymerase (Sso) having a wild-type amino acid sequence of SEQ ID NO: 9; and wherein:
the amino acid Y at position 519 of SEQ ID NO: 9 remains unchanged or is substituted with A, C, D, G, N, S, T or V;
the amino acid P at position 520 of SEQ ID NO: 9 remains unchanged or is substituted with A, C, G, I, L, M, N, S, T or V; and
the amino acid A at position 601 of SEQ ID NO: 9 is substituted with C, D, E, F, G, H, I, K, L, M, N, P, Q, R, T, V, W or Y.

20. The DNA polymerase variant of claim 1, wherein the DNA polymerase variant exhibits an activity of synthesizing nucleic acids in a template-independent manner by adding at least one nucleotide selected from the group of naturally occurring nucleotide, nucleotide analogue, or a mixture thereof, to an extendible initiator.

21. The DNA polymerase variant of claim 20, wherein the extendible initiator comprises a single-stranded oligonucleotide initiator, a blunt-ended double-stranded oligonucleotide initiator, or a mixture thereof.

22. The DNA polymerase variant of claim 20, wherein the extendible initiator is a free form nucleic acid to be reacted in a liquid phase.

23. The DNA polymerase variant of claim 20, wherein the extendible initiator is immobilized on a solid support, wherein the solid support comprises a particle, bead, slide, array surface, membrane, flow cell, well, microwell, nano-well, chamber, microfluidic chamber, channel, or microfluidic channel.

24. The DNA polymerase variant of claim 20, wherein the at least one nucleotide is linked with a detectable label.

25. The DNA polymerase variant of claim 20, wherein the at least one nucleotide comprises a ribose.

26. The DNA polymerase variant of claim 20, wherein the DNA polymerase variant exhibits the activity at reaction temperatures ranging from 10° C. to 100° C.

27. A kit for performing de novo enzymatic nucleic acid synthesis, comprising the DNA polymerase variant of claim 1, wherein the DNA polymerase variant exhibits activity of synthesizing nucleic acids in a template-independent manner by adding at least one nucleotide selected from the group of naturally occurring nucleotide, nucleotide analogue, or a mixture thereof, to an extendible initiator, thereby synthesizing a desired nucleic acid sequence.

28. A method for template-independent synthesis of an RNA oligonucleotide, comprising:
(a) providing an initiator oligonucleotide;
(b) providing the DNA polymerase variant of claim 1;
(c) combining the initiator oligonucleotide, the DNA polymerase variant, and one or more nucleotides under conditions sufficient for the addition of at least one nucleotide to the 3′ end of the initiator oligonucleotide.
